(19) United States
(12) Patent Application Publication
(10) Pub. No.: US 2008/0299182 A1
(43) Pub. Date: Dec. 4, 2008

(54) METHODS AND FORMULATIONS FOR TOPICAL GENE THERAPY

(76) Inventors: Shuyuan Zhang, Boyds, MD (US); Eric Onishi, Houston, TX (US)

Correspondence Address: FULBRIGHT & JAWORSKI L.L.P., 600 CONGRESS AVE., SUITE 2400, AUSTIN, TX, 78701, US

(21) Appl. No.: 12/040,520

(22) Filed: Feb. 29, 2008

Related U.S. Application Data
(60) Provisional application No. 60/892,427, filed on Mar. 1, 2007.

(51) Int. Cl. A61K 9/70; A61K 31/7088; A61P 35/04

(52) U.S. Cl. 424/443; 514/44

(57) ABSTRACT

Formulations of viral vectors for topical application are disclosed as well as methods for making the same. Also disclosed are methods of treating a subject or diagnosing disease in a subject using the formulations of the present invention.

This application claims the benefit of priority to U.S. provisional patent application Ser. No. 60/892,427, filed Mar. 1, 2007, the entire contents of which is hereby incorporated by reference.

### BACKGROUND OF THE INVENTION

1. Field of the Invention

The present invention relates generally to the field of pharmaceutical formulations and methods of making such. More particularly, the present invention relates to formulations of viral vectors for topical application to a subject.

2. Description of Related Art

Gene transfer is a relatively new modality that involves delivery of a particular gene particular target cells in a subject. Gene transfer for therapeutic purposes (i.e., gene therapy) involves the transfer of a therapeutic gene to target cells in a subject. Although the majority of gene therapy trials pertain to the treatment of cancer and vascular disease, gene therapy applications may include the treatment of single gene disorders or the detection of abnormal or hyperproliferative cells.

One aspect of successful gene therapy of cancer or other diseases is the ability to affect a significant fraction of the aberrant cells. Viral vectors are employed for this purpose. Recombinant adenoviruses have distinct advantages over retroviral and other gene delivery methods (reviewed in Siegfried, 1993). Adenoviruses have never been shown to induce tumors in humans and have been safely used as live vaccines (see Straus, 1984). Replication deficient recombinant adenoviruses can be produced by replacing the E1 region necessary for replication with the target gene. Adenovirus does not integrate into the human genome as a normal consequence of infection, thereby greatly reducing the risk of insertional mutagenesis. Stable, high titer recombinant adenovirus can be produced, allowing enough material to be produced to treat a large patient population. Moreover, adenovirus vectors are capable of highly efficient in vivo gene transfer into a broad range of tissue and tumor cell types.

Although viral vectors offer several advantages over other modes of gene delivery vehicles, they still exhibit some characteristics which impose limitations to their efficient use in vivo. These limitations primarily result in the limited ability of the vectors to efficiently deliver and target therapeutic genes to the aberrant cells. Attempts have been made to overcome this problem by direct injection of large quantities of viral vectors into the region containing the target cells. Current local administration of virus vectors is by injection of approximately 1×10¹² viral particles into the region of the target cells. Unfortunately, a high proportion of this material is not retained in the area of injection, but is quickly cleared through the circulatory and lymphatic systems, thus preventing infection of the target cells.

Besides virus-mediated gene-delivery systems, there are several non-viral options for gene delivery. One non-viral approach involves the use of liposomes to carry the therapeutic gene. Another approach, which is limited in application, is the direct introduction of therapeutic DNA into target cells.

Currently certain processes exist for topical delivery of pharmaceutical agents such as drugs. For example, U.S. Pat. No. 6,828,308, U.S. Pat. No. 6,280,752, U.S. Pat. No. 6,258,830, U.S. Pat. No. 5,914,334, U.S. Pat. No. 5,888,493, and U.S. Pat. No. 5,571,314 each pertain to the formulation of gels which could be used for drug delivery. Also, certain gel formulations specifically contemplated for topical use in the oral cavity may be found in U.S. patent application Ser. No. 11/336,664. Other delivery devices for drugs for topical use in the oral cavity also include devices such as film strips. An example of such a device is the Cool Mint Listerine PocketPaks® Strips, a micro-thin starch-based film impregnated with ingredients found in Listerine® Antiseptic (Thymol, Eucalyptol, Methyl Salicylate, Menthol). Non-active strip ingredients include pullulan, flavors, aspartame, potassium acesulfame, copper gluconate, polysorbate 80, carrageenan, glyceryl oleate, locust bean gum, propylene glycol and xanthan gum.

Additionally there are other known formulations for topical delivery of drugs in the form of transdermal patches. Formulations pertaining to transdermal or transcutaneous patches are discussed in detail, for example, in U.S. Pat. No. 5,770,219, U.S. Pat. No. 6,348,450, U.S. Pat. No. 5,783,208, U.S. Pat. No. 6,280,766 and U.S. Pat. No. 6,555,131.

In addition to topical delivery of drugs, certain processes exist that could be utilized for the topical delivery of proteins to the oral cavity. For example, U.S. patent application Ser. No. 10/741,861 discusses spherical protein particles which could be used for oral delivery. Additionally, U.S. patent application Ser. No. 10/383,266 discusses spherical protein or nucleic acid particles which can be used for oral delivery of antigens. However, it remains to be seen if any of these methods for the delivery of proteins or nucleic acids can be applied for the effective topical delivery of viral vectors.

Thus, there exists a need for new and improved compositions for the topical delivery of viral vector based gene therapy that can allow for the dissemination of such a vector and protect its potency during storage.

### SUMMARY OF THE INVENTION

The inventors have identified certain formulations of viral vectors that can be applied for the topical delivery of viral vectors to a subject. These formulations can be applied for the purpose of diagnosing or treating a disease, or storing viral vector. The viral vector may include, for example, a nucleic acid that encodes an agent that can be applied in the treatment of a hyperproliferative lesion in a subject, or a diagnostic agent that can be applied in diagnosing a hyperproliferative lesion in a subject. The formulation includes a viral vector and a biopolymer, and is configured in any manner suitable for topical application to a body surface, such as a mucosal surface.

Some embodiments of the present invention generally pertain to films, strips, or patches containing viral vectors and methods of making such films, strips, or patches. These films, strips, and patches allow for stable long term storage of viral vector. These new pharmaceutical formulations may allow for delivery of viral vectors to topical surfaces such as, but not limited to the oral mucosa and cervical mucosa.

A “strip” as used herein refers to a long narrow piece of material that includes a biopolymer and a viral vector. The strip may be formulated with an adhesive to facilitate adhesion to a surface, such as a mucosal surface. The strip may or may not be of uniform width.

A “film” as used herein refers to a strip that has elastic properties or is flexible. The film may be formulated to dissolve over time. A film may also be formulated with the addition of agents that are not therapeutic, such as sweetners or flavorants, for example, if the formulation is contemplated for oral application. In some embodiments, the film formulations of the invention adhere to mucosal surfaces (e.g., oral, vaginal, etc.) when wet.

As used herein, a “patch” is a piece of material or covering that can be applied to a surface of the body that includes a biopolymer and a viral vector, and that is not otherwise a strip or a film. The patch may be rectangular or in the shape of a square. It may be oval or circular. The surface of the body may be any body surface, such as a skin surface or a mucosal surface (e.g., the surface of the vagina or mouth). The patch can be composed of any material known to those of ordinary skill in the art.

Formulations pertaining to transdermal or transcutaneous films, strips, and patches are discussed in detail, for example, in U.S. Pat. No. 5,770,219 U.S. Pat. No. 6,348,450, U.S. Pat. No. 5,783,208, U.S. Pat. No. 6,280,766 and U.S. Pat. No. 6,555,131, each of which is herein specifically incorporated by reference into the specification.

In some embodiments, one or more additional therapeutic agents for the diagnosis, treatment and/or prevention of a disease can be included in the strips, films, or patches of the present invention. In addition, an impermeable backing layer may be incorporated to insure unidirectional flow of the drug, such as through a mucosal surface. In some cases a rate controlling film or membrane may also be laminated or sprayed onto the strip, film, or patch to further control the rate of release of viral vector.

Other aspects of the present invention concern methods of producing a film, strip, or patch containing a viral vector that involve casting a composition that includes a biopolymer and a viral vector into a mold. The composition that has been cast into the mold assumes the shape of a film, a strip, a patch, or other configuration suitable for application to a body surface, depending upon the configuration of the mold.

The composition may optionally further include one or more biopolymers, one or more polyols, one or more buffers, and one or more aqueous solvents. In some embodiments, the composition that has been cast into the mold is dried, wherein drying results in removal of some or all of the solvent from the composition that has been cast into the mold. For example, the composition that has been cast into the mold may be freeze-dried. Drying permits the composition that has been cast into the mold to assume the shape of the mold as a result of removal of solvent from the composition that was cast into the mold. The mold may be configured in any manner such that the composition that has been cast into the mold assumes a configuration suitable for application to a body surface, such as in the shape of a film, strip, or patch. Other formulations contemplated by the present invention include lozenges, discs, pellets, suppositories, and the like. In some embodiments, the composition is formulated to dissolve upon exposure to a certain pH or temperature.

In certain embodiments, the composition that includes a biopolymer and a viral vector is otherwise shaped into a film, strip, or patch without use of a mold. For example, the composition may be pressed into sheets, followed by cutting of the sheets into strips or patches. Drying may take place prior to, consecutively with, or following pressing of the composition into sheets.

Some embodiments of the methods set forth herein further include the step of removing the dried composition from the mold. The dried composition that is removed from the mold may be a film, strip, or patch, or may require further manipulation to be a film, strip, or patch. In some embodiments, the dried composition is removed from the mold, and is then subsequently cut into films, strips, or patches suitable for topical application to a subject.

The composition optionally include a lyoprotectant and/or a polyol, In particular embodiments, the composition further includes one or more aqueous solvents. For example, the aqueous solvent may be water or saline. The composition may also include one or more buffers. Examples of such buffers are discussed in the specification below. The method may further involve the step of removing aqueous solvent or buffer from the composition that has been cast into the mold. For example, removing may be by freeze-drying the composition in the mold to obtain a film, strip, or patch.

In other embodiments, the present invention relates to a film, strip, or patch for application of a viral vector to a subject comprising a biopolymer and a viral vector. In some embodiments, the film, strip, or patch further includes one or more of a lyoprotectant, a polyol, or a buffer. The film, strip, or patch may optionally be freeze-dried.

In specific embodiments the biopolymer is hydroxypropylmethyl cellulose, hydroxypropyl cellulose, sodium alginate, polyacrylate or a combination thereof. In a particular embodiments, the biopolymer is sodium alginate. In certain embodiments, prior to freeze-drying, the biopolymer is in a mixture of biopolymer and water wherein the biopolymer is in a concentration of about 0.1% to about 15% weight to volume. In a specific embodiment, prior to freeze-drying the biopolymer is in a concentration of about 1% to about 10% weight to volume. In a further embodiment, prior to free-drying the biopolymer is in a concentration of about 5% weight to volume.

The lyoprotectant may be any lyoprotectant known to those of ordinary skill in the art. For example, the lyoprotectant may be sucrose, fructose, glucose, galactose, mannose, sorbitol, trehalose, lactose, maltose, mannitol or a mixture thereof. In some embodiments, the lyoprotectant is sucrose. In certain embodiments, prior to freeze-drying the concentration of lyoprotectant is about 3% to about 20% weight to volume. In even more specific embodiments, the concentration of lyoprotectant is about 10% weight to volume.

The polyol may be any polyol known to those of ordinary skill in the art. Non-limiting examples of polyols include glycerol, propylene glycol, polyethylene glycol or a mixture thereof. In some embodiments, the polyol is glycerol. The polyol, for example, may be in a concentration of about 1% to about 30% weight to volume of the mixture or solution prior to freeze-drying.

In embodiments that include a buffer, the buffer may be any buffer known to those of ordinary skill in the art. Non-limiting examples of buffers include Tris-HCl, TES, HEPES, mono-Tris, brucine tetrahydrate, EPPS, tricine or histidine. In some embodiments the buffer is Tris-HCl. In further embodiments the buffer is included at a concentration of about 1 mM to about 50 mM.

The viral vector may be any viral vector known to those of ordinary skill in the art. Non-limiting examples include an adenoviral vector, an adeno-associated viral vector, a retroviral vector, a herpesviral vector or a poxviral vector. In particular embodiments, the viral vector is an adenoviral vector.

In certain embodiments the pharmaceutical formulation of a film or patch containing a viral vector has a titer of at least 80% of its starting titer after freeze drying. In other embodiments the pharmaceutical formulation containing the viral vector has a titer of at least 80% of the post freeze-drying titer after storage for one month.

In particular embodiments of the methods and pharmaceutical formulations of the present invention, the viral vector itself comprises a therapeutic nucleic acid. The therapeutic nucleic acid may be any therapeutic nucleic acid known to those of ordinary skill in the art. Other embodiments, the viral vector comprises a diagnostic nucleic acid. The diagnostic nucleic acid may be any such nucleic acid known to those of ordinary skill in the art.

In certain embodiments, the therapeutic nucleic acid encodes a tumor suppressor. Non-limiting examples of tumor suppressors include MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a SEM A3 polypeptide. In particular embodiments the tumor suppressor is p53, MDA-7 or FUS1.

In embodiments wherein the therapeutic nucleic acid encodes a tumor antigen, the tumor antigen may be any tumor antigen. Non-limiting examples include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, ING1, mamaglobin, cyclin B1, S100, BRCA1, BRCA2, a tumor immunoglobulin idiotype, a tumor T-cell receptor clonotype, MUC-1, or epidermal growth factor receptor.

In some embodiments, the viral vector comprises a diagnostic nucleic acid. A “diagnostic nucleic acid” is a nucleic acid that is known or suspected to be of benefit in identifying the presence or absence of a disease or health-related condition, or that is known or suspected to be of benefit in identifying a subject at risk of developing a particular disease or health-related condition. Also included in the definition of “diagnostic nucleic acid” is a nucleic acid sequence that encodes one or more reporter proteins. A “reporter protein” refers to an amino acid sequence that, when present in a cell or tissue, is detectable and distinguishable from other genetic sequences or encoded polypeptides present in cells. A reporter protein may be a naturally occurring protein or a protein that is not naturally-occurring. If naturally-occurring, it may be detectable as a result of the amount of expression following gene transfer, or it may be a protein to which a detectable tag can be attached. Non-limiting examples of such reporter proteins include fluorescent proteins such as green fluorescent protein (gfp), cyan fluorescent protein (cfp), red fluorescent protein (rfp), or blue fluorescent protein (bfp), or derivatives of these proteins, or enzymatic proteins such as β-galactosidase, chemilluminesent proteins such as luciferase, somatostatin receptor amino acid sequence, a sodium iodide symporter amino acid sequence, a luciferase amino acid sequence, and a thymidine kinase amino acid sequence.

In certain embodiments of the present invention, the therapeutic or diagnostic nucleic acid of the viral vector is comprised in an expression cassette. The expression cassette may itself comprise a promoter operatively coupled to the nucleic acid, wherein the promoter is active in cells of a subject.

Also included in the embodiments of the present invention are methods of detecting, treating or preventing disease in a subject comprising administering a film, strip, or patch of the present invention. In particular embodiments, the nucleic acid encodes a fluorescent protein or is a diagnostic nucleic acid encoding a reporter protein. In such embodiments, the methods of the present invention pertain to detecting a lesion in a subject. The lesion may be a hyperproliferative lesion, such as cancer. In other embodiments, the nucleic acid encodes a tumor suppressor and the method is further defined as a method of treating a subject with a hyperproliferative disease. In such embodiments the tumor suppressor may be any tumor suppressor, such as those described above. In some embodiments, the method is a method of diagnosing and treating a hyperproliferative disease in a subject.

The subject may be any subject. In particular embodiments, the subject is a mammal. Examples of mammals include mice, rats, rabbits, dogs, cats, cows, horses, sheep, goats, non-human primates (such as monkeys, chimpanzees, and baboons), and humans. In specific embodiments, the subject is a human. For example, the human may be a patient with a hyperproliferative disease, or a patient who is suspected of having a hyperproliferative disease.

Other embodiments of the present invention include pharmaceutical compositions that include (i) a biopolymer; (ii) a lyoprotectant; (iii) a polyol; (iv) a buffer; and (v) a viral vector. The biopolymer, lyoprotectant, polyol, buffer, and viral vector can be any of those set forth above and elsewhere in this specification. The composition may optionally include additional agents, such as an aqueous solvent or pharmaceutical carrier. The compositions set forth herein may include more than one biopolymer, lyoprotectant, polyol, buffer, or viral vector.

The present invention also contemplates kits that include a film, strip, or patch of the present invention in a sealed container.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Picture of the Freeze Dried Film. Six-well plate after freeze-drying demonstrating the films formed. Wells 1 and 2: Negative control of 1% sodium alginate and 5% sucrose only (1.5 ml per well). Wells 3 and 4: 1% sodium alginate and 5% sucrose with 1×10¹¹ vp/ml of Ad-GFP (1.5 ml per well). Wells 5 and 6: 1% sodium alginate and 5% sucrose with 1×10¹⁰ vp/ml of Ad-GFP (1.5 ml per well).

FIG. 2A. 293 Cell Transduction with 1×10¹¹ vp/ml Ad-GFP Film. Left Column demonstrates a section of the 293 cells in normal light showing the cells outside of the film, the film border and the cells underneath the film. Right column demonstrates the GFP expression of the 293 cells under fluorescent light.

FIG. 2B. 293 Cell Transduction with 1×10¹⁰ vp/ml Ad-GFP Film. Left Column demonstrates a section of the 293 cells under fluorescent light outside the film while the right column depicts the 293 cells under the same conditions which were underneath the film. The absence of GFP expression in the right column is likely due to lack of oxygenation of the cells during transduction.

FIG. 2C. 293 Cell Transduction with Biopolymer Control Film. Left Column demonstrates a section of the 293 cells under fluorescent light outside the film while the right column depicts the 293 cells under the same conditions which were underneath the film. No GFP expression is observed under either condition.

FIG. 3. Freeze-Dried Film Stability Assay. 293 cells were transduced with Ad-GFP film which had been stored for 1 or 2 months at −20° C. Right column depicts GFP expression in transduced 293 cells as viewed by fluorescence microscope.

FIG. 4A. Transduction Efficiency Based on Contact Time. Illustration depicts placement of Ad-GFP Film on 293 cells to determine transduction efficiency based on contact time.

FIG. 4B. Transduction Efficiency Based on Contact Time. 293 cells were exposed to Ad-GFP film for a period of time of 15 minutes, 30 minutes, 1 hour and 2 hours. Left column depicts 293 cells as viewed under normal light microscope. Right column depicts 293 cells as viewed under fluorescence microscope.

FIG. 5. Transduction Efficiency of Oral Epithelial Model. Ad-GFP film was placed on apical surface of EpiOral™ oral epithelial model. Left column indicates number of hours post Ad-GFP film exposure before cell observation under fluorescence microscope (middle column) or normal light microscope (right column).

FIG. 6A. Ad-GFP Labeling of Tumor Cells in Oral Epithelial Model. Illustration depicts placement of the EpiOral™ oral epithelial model in a 6 well plate with H1299 cancer cells.

FIG. 6B. Differential Expression of Ad-GFP Transduced H1299 Cells as Compared to EpiOral™ Cells. Pictures depict EpiOral™ cells, H1299 cells and a combination of both under normal light microscope (left column) and fluorescence light microscope (right column) 24 hours after Ad-GFP transduction.

### DESCRIPTION OF ILLUSTRATIVE EMBODIMENTS

The inventors have identified certain formulations of viral vectors that can be used in the diagnosis, treatment, and/or prevention of disease in a subject. The formulations, such as films, strips, or patches, can be used for extended, localized application of gene therapy vectors to a variety of topical surfaces to achieve better bioavailability and therapeutic effect. These compositions include a biopolymer and a viral vector. The compositions optionally include a lyoprotectant. Such compositions are formulated for application to a body surface of a subject, such as a tumor bed after surgery, the oral cavity or a mucosal service. The novel compositions and methods set forth herein can be applied in the detection, prevention or treatment of any of a number of diseases and health-related conditions, so long as the application is topical. Examples of such diseases which may be treated with viral vector based gene therapy include cancer and infections. Applications of these novel compositions in the diagnosis, treatment, and prevention of disease represent an improvement in existing gene therapy technology.

### A. PHARMACEUTICAL COMPOSITIONS

### 1. Definitions

The phrase “pharmaceutical composition” and “formulated” refer to molecular entities and compositions that do not produce an adverse, allergic or other untoward reaction when administered to a mammal or human, as appropriate. As used herein, a “pharmaceutical composition” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients. For instance, the a composition for topical delivery to the oral cavity may include may include a flavorant or the composition may contain supplementary ingredients to make the formulation timed-release. Formulations are discussed in greater detail in the following sections.

Some of the pharmaceutical composition of the present invention are formulated for oral delivery. Oral delivery includes administration via the mouth of an animal or other mammal, as appropriate. Oral delivery also includes topical administration to any part of the oral cavity, such as to the gums, teeth, oral mucosa, or to a lesion in the mouth, such as a pre-neoplastic or neoplastic lesion. Oral delivery also includes delivery to a mouth wound or a tumor bed in the mouth.

In the context of the present invention, “topical administration” is defined to include administration to a surface of the body such as the skin, oral mucosa, gastrointestinal mucosa, eye, anus, cervix or vagina, or administration to the surface of the bed of an excised lesion in any of these areas (i.e., the surgical bed of an excised pharyngeal HNSCC or an excised cervical carcinoma), or administration to the surface of a hollow viscus, such as the bladder.

### 2. Film, Strips, and Patches

Films, strips, and patches have been used for topical delivery of a number of small molecule drugs. In order to form the film or patch, organic solvent or hot melt extrusion methods are generally used. These methods involve harsh conditions that cannot be applied for fragile gene therapy vectors. Tor form a film or patch the vectors need to be dried. It is known from our own experience that viral vectors are extremely sensitive to air drying. Unfortunately, there have been no reported studies on the preparation of a gene therapy film for topical application in the literature. Therefore, such a patch or film was generated for this purpose.

In certain embodiments of the present invention, the film strip will comprise gene therapy vectors, biopolymers, lyoprotectants and optionally excipients. Certainly, other components may also be contemplated so long as they are within the spirit of the scope of the present invention.

a. Biopolymers

Biopolymers may be generally classified as natural polymers. Examples of biopolymers include poly-acrylic acid, poly-cyanoacrylates, polypeptides, poly-anhydrides, poly-depsipeptide, poly-esters such as poly-lactic acid or PLA, poly lactic-co-glycolic acid or PLGA, poly β-hydroxybutryate, poly-caprolactone, poly-dioxanone; polyethylene glycol, poly-hydroxypropylmethylacrylamide, polyvinyl alcohol, polyvinylpyrrolidone, albumin, alginate such as sodium alginate, cellulose and cellulose derivatives such as hydroxypropylmethyl cellulose and hydroxypropyl cellulose, collagen, fibrin, gelatin, hyaluronic acid, oligosaccarides, glycaminoglycans, sulfated polysaccarides, blends and copolymers thereof.

b. Lyoprotectants

Lyoprotectants are chemicals designed to preserve and protect during the process of drying. Lyoprotectants include sugars such as sucrose, fructose, glucose, galactose, mannose, sorbitol, trehalose, lactose and maltose; polyols such as mannitol; amino acids such as glycine, histidine, leucine, threonine, arginine, and lysine, and polymers such as polyvinyl pyrrolidone. Other lyoprotectants include dextran, and hydroxypropyl-beta-cyclodextrin.

c. Excipients

In addition to the functional biopolymers and lyoprotectants, a film or patch, such as a film or patch for topical administration to the oral cavity may also include other excipients. Examples may include glycerin, PEG, hydrated silica, xanthum gum, glycan carbomer 956, Tween 80, fluoride, carrageenan, an adhesive, or a flavorant.

d. Additional Aspects

In the embodiments of the present invention, a liquid or colloidal or gel-type mixture or solution of the film strip ingredients will be cast in a mold prior to the freeze-drying process. The freeze-drying may take place in or out of the mold. The mold will be of a design such that the liquid, colloidal or gel-type mixture can be placed on or in the mold, wherein after the freeze-drying process, the mixture will be in a solid form.

In certain embodiments the mold may posses a high surface area to height ratio such as a person of skill in the art would find on a baking tray used in a kitchen or on a Petri dish used in a laboratory setting. Such a mold may be used such that after freeze-drying, the mixture will be in the form of a film. In certain embodiments the freeze-dried film may be cut into specific sizes.

In other embodiments, the mold may be formed to have several smaller compartments such that several pre-sized films or patches are formed after freeze-drying. Non limiting examples of molds of this type include 6 and 12 well plates or ice cube trays. One of skill in the art would be familiar with various molds having several smaller compartments.

In still another embodiment, the mold may have a low surface area to height ratio, such that after freeze-drying, the mixture is in a substantially three dimensional form. For example, the freeze-dried mixture may be in the form of a cylinder, a rectangle or a cube. In such a solid form, one of skill in the art would cut or slice the mixture into the desired film size and thickness. A non-limiting example of an instrument designed to slice three dimensional objects into thin films is a microtome, an instrument commonly used in the preparation of tissue sample slides for microscopy.

### B. VIRAL GENE THERAPY VECTORS

A viral vector is a virus that can transfer genetic material from one location to another, such as from the point of application to a target cell of interest. In certain embodiments of the present invention, the nucleic acids of the compositions set forth herein is a “naked” nucleic acid sequence, which is not comprised in a viral vector or delivery agent, such as a lipid or liposome. In other embodiments of the present invention, however, the nucleic acid is comprised in a viral vector. A “viral vector” is meant to include those constructs containing viral sequences sufficient to (a) support packaging of an expression cassette comprising the therapeutic nucleic acid sequences and (b) to ultimately express a recombinant gene construct that has been cloned therein. One of ordinary skill in the art would be familiar with the various types of viruses that are available for use as vectors for gene delivery to a target cell of interest. Each of these is contemplated as a vector in the present invention. Exemplary vectors are discussed below.

### 1. Adenoviral Vectors

The pharmaceutical compositions and methods of the present invention may involve expression constructs of the therapeutic nucleic acids comprised in adenoviral vectors for delivery of the nucleic acid. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both non-dividing and dividing cells and can be produced in large quantities. In many of the clinical trials for cancer, local intratumoral injections have been used to introduce the vectors into sites of disease because current vectors do not have a mechanism for preferential delivery to tumor. In vivo experiments have demonstrated that administration of adenovirus vectors systemically resulted in expression in the oral mucosa (Clayman et al., 1995). Topical application of Ad-βgal and Ad-p53-FLAG on organotypic raft cultures has demonstrated effective gene transduction and deep penetration through multiple cell layers (Eicher et al., 1996). Therefore, gene transfer strategy using the adenoviral vector is potentially feasible in patients at risk for lesions and malignancies involving genetic alterations in p53.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

### 2. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

### 3. AAV Vectors

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte and Carter, 1995).

### 4. Herpesvirus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of α genes, the first set of genes to be expressed after infection, is enhanced by the virion protein number 16, or α-transinducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

### 5. Vaccinia Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

### 6. Oncolytic Viral Vectors

Oncolytic viruses are also contemplated as vectors in the present invention. Oncolytic viruses are defined herein to generally refer to viruses that kill tumor or cancer cells more often than they kill normal cells. Exemplary oncolytic viruses include adenoviruses which overexpress ADP. These viruses are discussed in detail in U.S. patent application Ser. No. 10/810,063, U.S. Pat. No. 6,627,190, and U.S. patent application Ser. No. 09/351,778, each of which is specifically incorporated by reference in its entirety into this section of the application and all other sections of the application. Exemplary oncolytic viruses are discussed elsewhere in this specification. One of ordinary skill in the art would be familiar with other oncolytic viruses that can be applied in the pharmaceutical compositions and methods of the present invention.

### 7. Other Viral Vectors

Other viral vectors that may be employed as vectors in the present invention include those viral vectors that can be applied in vaccines, or in dual vaccine and immunotherapy applications. Viral vectors, and techniques for vaccination and immontherapy using viral vectors, are described in greater detail in PCT application WO0333029, WO0208436, WO0231168, and WO0285287, each of which is specifically incorporated by reference in its entirely for this section of the application and all other sections of this application. Additional vectors that can be applied in the techniques for vaccination and dual immunotherapy/vaccination include those oncolytic viruses set forth above.

Other viral vectors also include baculovirus vectors, parvovirus vectors, picornavirus vectors, alphavirus vectors, semiliki forest virus vectors, Sindbis virus vectors, lentivirus vectors, and retroviral vectors. Vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Other viral vectors for application in the compositions and methods of the present invention include those vectors set forth in Tang et al., 2004, which is herein specifically incorporated by reference in its entirety for this section of the application and all other sections of the application.

### C. NUCLEIC ACIDS ENCODED BY VIRAL GENE THERAPY VECTORS

### 1. Therapeutic Nucleic Acids

In some embodiments of the patch or strip pharmaceutical formulations set forth herein, the nucleic acid is a therapeutic nucleic acid. A “therapeutic nucleic acid” is defined herein to refer to a nucleic acid which can be administered to a subject for the purpose of treating or preventing a disease. The nucleic acid is one which is known or suspected to be of benefit in the treatment of a disease or health-related condition in a subject. Diseases and health-related conditions are discussed at length elsewherein this this specification.

Therapeutic benefit may arise, for example, as a result of alteration of expression of a particular gene or genes by the nucleic acid. Alteration of expression of a particular gene or genes may be inhibition or augmentation of expression of a particular gene. In certain embodiments of the present invention, the therapeutic nucleic acid encodes one or more proteins or polypeptides that can be applied in the treatment or prevention of a disease or health-related condition in a subject. The terms “protein” and “polypeptide” are used interchangeably herein. Both terms refer to an amino acid sequence comprising two or more amino acid residues.

Any nucleic acid known to those of ordinary skill in the art that is known or suspected to be of benefit in the treatment or prevention of a disease or health-related condition is contemplated by the present invention as a therapeutic nucleic acid. The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein. In some embodiments, the nucleic acid includes a therapeutic gene. The term “gene” is used to refer to a nucleic acid sequence that encodes a functional protein, polypeptide, or peptide-encoding unit.

As will be understood by those in the art, the term “therapeutic nucleic acid” includes genomic sequences, cDNA sequences, and smaller engineered gene segments that express, or may be adapted to express, proteins, polypeptides, domains, peptides, fusion proteins, and mutants. The nucleic acid may comprise a contiguous nucleic acid sequence of about 5 to about 12000 or more nucleotides, nucleosides, or base pairs.

Encompassed within the definition of “therapeutic nucleic acid” is a “biologically functional equivalent” of a therapeutic nucleic acid that has proved to be of benefit in the treatment or prevention of a disease or health-related condition. Accordingly, sequences that have about 70% to about 99% homology to a known nucleic acid are contemplated by the present invention.

### 2. Nucleic Acids that Encode Tumor Suppressors and Pro-Apoptotic Proteins

In some embodiments, the nucleic acid of the claimed pharmaceutical compositions include a nucleic acid sequence that encodes a protein or polypeptide that can be applied in the treatment or prevention of cancer or other hyperproliferative disease. Examples of such proteins include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, IL-13, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAl, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

A “tumor suppressor” refers to a polypeptide that, when present in a cell, reduces the tumorigenicity, malignancy, or hyperproliferative phenotype of the cell. The nucleic acid sequences encoding tumor suppressor gene amino acid sequences include both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

A nucleic acid encoding a tumor suppressor generally refers to a nucleic acid sequence that reduce the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressors include, but are not limited to, MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes at www.cise.ufl.edu/˜yyl/HTML-TSGDB/Homepage.html. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

A nucleic acid encoding a pro-apoptotic protein encode a protein that induces or sustains apoptosis to an active form. The present invention contemplates inclusion of any nucleic acid encoding a pro-apoptotic protein known to those of ordinary skill in the art. Exemplary pro-apoptotic proteins include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic proteins, including those not specifically set forth herein.

Nucleic acids encoding pro-apoptotic amino acid sequences include, for example, cDNAs, cRNAs, mRNAs, and subsequences thereof encoding active fragments of the respective pro-apoptotic amino acid sequence.

One of ordinary skill in the art would understand that there are other nucleic acids encoding proteins or polypeptides that can be applied in the treatment of a disease or health-related condition that are not specifically set forth herein. Further, it is to be understood that any of the therapeutic nucleic acids mentioned elsewhere in this specification, such as nucleic acids encoding cytokines, may be applied in the treatment and prevention of cancer.

### 3. Nucleic Acids Encoding Cytokines

In some embodiments of the pharmaceutical compositions set forth herein the nucleic acid encodes a cytokine. The term “cytokine” is a generic term for proteins released by one cell population which act on another cell as intercellular mediators. The nucleic acid sequences may encode the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factors (FGFs) such as FGF-α and FGF-β; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-α; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand, FLT-3 or MDA-7.

A non limiting example of growth factor cytokines involved in wound healing include: epidermal growth factor, platelet-derived growth factor, keratinocyte growth factor, hepatycyte growth factor, transforming growth factors (TGFs) such as TGF-α and TGF-β, and vascular endothelial growth factor (VEGF). These growth factors trigger mitogenic, motogenic and survival pathways utilizing Ras, MAPK, PI-3K/Akt, PLC-gamma and Rho/Rac/actin signaling. Hypoxia activates pro-angiogenic genes (e.g., VEGF, angiopoietins) via HIF, while serum response factor (SRF) is critical for VEGF-induced angiogenesis, re-epithelialization and muscle restoration. EGF, its receptor, HGF and Cox2 are important for epithelial cell proliferation, migration re-epithelializaton and reconstruction of gastric glands. VEGF, angiopoietins, nitric oxide, endothelin and metalloproteinases are important for angiogenesis, vascular remodeling and mucosal regeneration within ulcers. (Tamawski, 2005)

### 4. Nucleic Acids Encoding Enzymes

Other examples of therapeutic nucleic acids include nucleic acids encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. However, in certain embodiments of the invention, it is contemplated that the invention specifically does not include one or more of the enzymes identified above or in the following paragraph.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, glucosyltransferase, HSV thymidine kinase, or human thymidine kinase.

A therapeutic nucleic acid of the present invention may encode a superoxide dismutase (SOD). SOD, which exists in several isoforms, is a metalloenzyme which detoxifies superoxide radicals to hydrogen peroxide. Two isoforms are intracellular: Cu/Zn-SOD, which is expressed in the cytoplasm, and Mn-SOD, which is expressed in mitochondria (Linchey and Fridovich, 1997). Mn-SOD has been demonstrated to increase resistance to radiation in hematopoetic tumor cell lines transfected with MnSOD cDNA (Suresh et al., 1993). Adenoviral delivery of Cu/Zn-SOD has been demonstrated to protect against ethanol induced liver injury (Wheeler et al., 2001). Additionally adenoviral mediated gene delivery of both Mn-SOD and Cu/Zn-SOD are equally efficient in protection against oxidative stress in a model of warm ischemia-reprofusion (Wheeler et al., 2001).

### 5. Nucleic Acids Encoding Hormones

Therapeutic nucleic acids also include nucleic acids encoding hormones. Examples include, but are not limited to, growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, and thyrotropin releasing hormone.

### 6. Nucleic Acids Encoding Antigens

The pharmaceutical compositions set forth herein may include a nucleic acid that encodes one or more antigens. For example, the therapeutic gene may encode antigens present in tumors, pathogens, or immune effectors involved in autoimmunity. These genes can be applied, for example, in formulations that would be applied in vaccinations for immune therapy or immune prophylaxis of neoplasias, infectious diseases and autoimmune diseases.

a. Tumor Antigens

In certain embodiments, the therapeutic nucleic acid encodes a tumor antigen. Tumor antigens are well-known to those of ordinary skill in the art. Examples include, but are not limited to, those described by Dalgleish (2004), Finn (2003), and Hellstrom and Helstrom (2003), each of which is herein incorporated by reference in its entirety. A non-limiting list of tumor antigens includes: MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, ING1, mamaglobin, cyclin B1, S100, BRCA1, BRCA2, a tumor immunoglobulin idiotype, a tumor T-cell receptor clonotype, MUC-1, or epidermal growth factor receptor. Other examples can be found on http://www.bioinfo.org.cn/hptaa/search.php, which is herein specifically incorporated by reference.

b. Microorganism Antigens

In some embodiments, the nucleic acid encodes a microorganism antigen. The term “microorganism” includes viruses, bacteria, microscopic fungi, protozoa and other microscopic parasites. A “microorganism antigen” refers to a polypeptide that, when presented on the cell surface by antigen presenting cells (APCs), induces an immune response. This response may include a cytotoxic T cell response or the production of antibodies or both.

Examples of viruses from which microorganism antigens may be derived include: human herpes viruses (HHVs)-1 through 8; herpes B virus; HPV-16, 18, 31, 33, and 45; hepatitis viruses A, B, C, δ; poliovirus; rotavirus; influenza; lentiviruses; HTLV-1; HTLV-2; equine infectious anemia virus; eastern equine encephalitis virus; western equine encephalitis virus; venezuelan equine encephalitis virus; rift valley fever virus; West Nile virus; yellow fever virus; Crimean-Congo hemorrhagic fever virus; dengue virus; SARS coronavirus; small pox virus; monkey pox virus and/or the like.

Examples of viral microorganisms include, but are not limited to: retroviridae, flaviridae, coronaviridae, picornaviridae, togaviridae, rhabdoviridae, paramyxoviridae, orthomyxoviridae, bunyaviridae, arenaviridae, reoviridae, polyomaviridae, papillomaviridae, herpesviridae and hepadnaviridae.

Examples of retroviridae include lentiviruses such as HIV-1, HIV-2, SIV, FIV, Visna, CAEV, BIV and EIAV. Genes encoded by lentiviruses may include gag, pol, env, vif, vpr, vpu, nef, tat, vpx and rev. Other examples of retroviruses include alpha retroviruses such as avian leukosis virus, avian myeloblastosis virus, avian sarcoma virus, fujinami sarcoma virus and rous sarcoma virus. Genes encoded by alpha retroviruses may include gag, pol and env. Further examples of retroviruses include beta retroviruses such as jaagsiekte sheep retrovirus, langur virus, Mason-Pfizer monkey virus, mouse mammary tumor virus, simian retrovirus 1 and simian retrovirus 2. Genes encoded by beta retroviruses may include gag, pol, pro and env. Still further examples of retroviruses include delta retroviruses such as HTLV-1, HTLV-2, bovine leukemia virus, and baboon T cell leukemia virus. Genes encoded by delta retroviruses may include gag, pol, env, tax and rex. Still further examples of retrovirus include spumaviruses such as bovine, feline, equine, simian and human foamy viruses. Genes encoded by spumaviruses may include gag, pol, env, bel-1, bel-2 and bet.

Examples of flaviridae include but are not limited to: hepatitis C virus, mosquito borne yellow fever virus, dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, louping III virus, Powassan virus, Omsk hemorrhagic fever virus, the genus rubivirus (rubella virus) and the genus pestivirus (mucosal disease virus, hog cholera virus, border disease virus). Genes encoded by flaviviruses include the flavivirus polyprotein from which all flavivirus proteins are derived. Nucleic acid sequences encoding the flavivirus polyprotein may include sequences encoding the final processed flavivirus protein products such as C, prM, E, NS1, NS2A, NS2B, NS3, NS4A, NS4B and NS5.

Examples of coronaviridae include but are not limited to: human respiratory coronaviruses such as SARS and bovine coronaviruses. Genes encoded by coronaviridae may include pol, S, E, M and N.

Examples of picornaviridae include but are not limited to the genus Enterovirus (poliovirus, Coxsackie virus A and B, enteric cytopathic human orphan (ECHO) viruses, hepatitis A virus, simian enteroviruses, murine encephalomyelitis (ME) viruses, poliovirus muris, bovine enteroviruses, porcine enteroviruses, the genus cardiovirus (encephalomyocarditis virus (EMC), mengovirus), the genus rhinovirus (human rhinoviruses including at least 113 subtypes; other rhinoviruses) and the genus apthovirus (foot and mouth disease (FMDV). Genes encoded by picornaviridae may include the picornavirus polyprotein. Nucleic acid sequences encoding the picornavirus polyprotein may include sequences encoding the final processed picornavirus protein products such as VPg, VPO, VP3, VP1, 2A, 2B, 2C, 3A, 3B, 3C and 3D.

Examples of togaviridae include but are not limited to including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis Eastern equine encephalitis virus). Examples of genes encoded by togaviridae may include genes coding for nsP1, nsP2, nsP3 nsP4, C, E1 and E2.

Examples of rhabdoviridae include, but are not limited to: including the genus vesiculovirus (VSV), chandipura virus, Flanders-Hart Park virus) and the genus lyssavirus (rabies virus). Examples of genes encoded by rhabdoviridae may include N, P, M, G, and L.

Examples of filoviridae include Ebola viruses and Marburg virus. Examples of genes encoded by filoviruses may include NP, VP35, VP40, GP, VP35, VP24 and L. Examples of paramyxoviruses include, but are not limited to: including the genus paramyxovirus (parainfluenza virus type 1, sendai virus, hemadsorption virus, parainfluenza viruses types 2 to 5, Newcastle disease Virus, mumps virus), the genus morbillivirus (measles virus, subacute sclerosing panencephalitis virus, distemper virus, Rinderpest virus), the genus pneumovirus (respiratory syncytial virus (RSV), bovine respiratory syncytial virus and pneumonia virus of mice). the family paramyxoviridae, including the genus Paramyxovirus (Parainfluenza virus type 1, Sendai virus, hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice). Examples of genes encoded by paramyxoviridae may include N, PIC/V, P/C/V/R, M, F, HN, L, V/P, NS1, NS2, SH and M2.

Examples of orthomyxoviridae include influenza viruses. Examples of genes encoded by orthomyxoviridae may include PB1, PB2, PA, HA, NP, NA, M1, M2, NS1 and NS2.

Examples of bunyaviruses include, but are not limited to: the genus bunyvirus (bunyamwera and related viruses, California encephalitis group viruses), the genus phlebovirus (sandfly fever Sicilian virus, Rift Valley fever virus), the genus nairovirus (Crimean-Congo hemorrhagic fever virus, Nairobi sheep disease virus) and the genus uukuvirus (uukuniemi and related viruses). Examples of genes encoded by bunyaviruses may include N, G1, G2 and L.

Examples of arenaviruses include, but are not limited to: lymphocytic choriomeningitis virus, lassa fever virus, Argentine hemorrhagic fever virus, Bolivian hemorrhagic fever virus and Venezuelan hemorrhagic fever virus. Examples of genes encoded by arenaviruses may include NP, GPC, L and Z.

Examples of reoviruses include, but are not limited to: the genus orthoreovirus (multiple serotypes of both mammalian and avian retroviruses), the genus orbivirus (Bluetongue virus, Eugenangee virus, Kemerovo virus, African horse sickness virus, and Colorado Tick Fever virus) and the genus rotavirus (human rotavirus, Nebraska calf diarrhea virus, murine rotavirus, simian rotavirus, bovine or ovine rotavirus, avian rotavirus). Examples of genes encoded by reoviruses may include genome segments named for their corresponding protein products, such as VP1, VP2, VP3, VP4, NSP1, NSP3, NSP2, VP7, NSP4, NSP5 and NSP6.

Examples of polyomaviridae include, but are not limited to BK and JC viruses. Examples of genes encoded by polyomaviruses may include Agno, P2, VP3, VP2, VP1, large T and small t.

Examples of papillomaviridae include, but are not limited to: HPV-16 and HPV-18. Examples of genes encoded by papillomaviruses may include E1, E2, E3, E4, E5, E6, E7, E8, L1 and L2.

Examples of herpesviridae include, but are not limited to: Human Herpes Virus (HHV) 1, HHV2, HHV3, HHV4, HHV5, HHV6, HHV7 and HHV8. Examples of genes encoded by herpesviruses may include γ₁34.5, ORF P, ORFO, αO, U_{L}1 through U_{L}56, α4, α22, U_{S}2 through U_{S}12, Ori_{S}TU and LATU.

Examples of hepadnaviruses include but is not limited to hepatitis B virus. Examples of genes encoded by hepadnaviruses may include S, C, P and X.

Examples of fungi from which microorganism antigens may be derived include: histoplasma capsulatum; aspergillus; actinomyces; candida, streptomyces and/or the like.

Examples of protozoa or other microorganisms from which antigens may be derived include plasmodium falciparum, plasmodium vivax, plasmodium ovale, plasmodium malariae, and the like. Genes derived from plasmodium species may include PyCSP, MSP1, MSP4/5, Pvs25 and Pvs28.

Examples of bacteria from which microorganism antigens may be derived include: mycobacterium tuberculosis; yersinia pestis; rickettsia prowazekii; rickettsia rickettsii; francisella tularensis; bacillus anthracis; helicobacter pylori; salmonella typhi; borrelia burgdorferi; streptococcus mutans; and/or the like. Genes derived from mycobacterium tuberculosis may include 85A, 85B, 85C and ESAT-6. Genes derived from yersinia pestis may include lcrV and cafl. Genes derived from rickettsia species may include ospA, invA, ompA, ompB, virB, cap, tlyA and tlyC. Genes derived from francisella tularensis may include nucleoside diphosphate kinase, isocitrate dehydrogenase, Hfq and ClpB. Genes derived from bacillus anthracis may include PA, BclA and LF. Genes derived from helicobacter pylori may include hpaA, UreB, hspA, hspB, hsp60, VacA, and cagE. Genes derived from salmonella typhi may include mpC, aroC, aroD, htrA and CS6. Genes derived from borrelia burgdorferi may include OspC.

Examples of fungi from which microorganism antigens may be derived include: hitoplasma; ciccidis; immitis; aspargillus; actinomyces; blastomyces; candida, streptomyces and/or the like.

Examples of protozoa or other microorganisms from which antigens may be derived include: plasmodium falciparum; plasmodium vivax; plasmodium ovale; plasmodium malariae; giadaria intestinalis and/or the like.

The microorganism antigen may be a glucosyltransferases derived from Streptococci mutans. The glucosyltransferases mediate the accumulation of S. mutans on the surface of teeth. Inactivation of glucosyltransferase has been demonstrated to cause a reduction in dental caries (Devulapalle and Mooser, 2001).

Another example an antigen derived from Streptococci mutans is PAc protein. PAc is a 190-kDa surface protein antigen involved in the colonization of Streptococci mutans, which mediates the initial adherence of this organism to tooth surfaces. Recently, it has been reported that in vivo administration of plasmid DNA encoding a fusion protein of amino acid residues 1185-1475 encoded by the glucosyltransferase-B of S. mutans, and amino acid residues 222-965 encoded by the PAc gene of S. mutans elicited an immune response against these respective gene products (Guo et al., 2004).

### 7. Nucleic Acids Encoding Antibodies

The nucleic acids set forth herein may encode an antibody. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

In certain embodiments of the present invention, the nucleic acid of the pharmaceutical compositions set forth herein encodes a single chain antibody. Single-chain antibodies are described in U.S. Pat. Nos. 4,946,778 and 5,888,773, each of which are hereby incorporated by reference.

### 8. Diagnostic Nucleic Acids

The pharmaceutical compositions of the present invention may include a nucleic acid that is a diagnostic nucleic acid. A “diagnostic nucleic acid” is a nucleic acid that can be applied in the diagnosis of a disease or health-related condition. Also included in the definition of “diagnostic nucleic acid” is a nucleic acid sequence that encodes one or more reporter proteins. A “reporter protein” refers to an amino acid sequence that, when present in a cell or tissue, is detectable and distinguishable from other genetic sequences or encoded polypeptides present in cells. In some embodiments, a therapeutic gene may be fused to the reporter or be produced as a separate protein. For example, the gene of interest and reporter may be induced by separate promoters in separate delivery vehicles by co-transfection (co-infection) or by separate promoters in the same delivery vehicle. In addition, the two genes may be linked to the same promoter by, for example, an internal ribosome entry site, or a bi-directional promoter. Using such techniques, expression of the gene of interest and reporter correlate. Thus, one may gauge the location, amount, and duration of expression of a gene of interest. The gene of interest may, for example, be an anti-cancer gene, such as a tumor suppressor gene or pro-apoptotic gene.

Because cells can be transfected with reporter genes, the reporter may be used to follow cell trafficking. For example, in vitro, specific cells may be transfected with a reporter and then returned to an animal to assess homing. In an experimental autoimmune encephalomyelitis model for multiple sclerosis, Costa et al. (2001) transferred myelin basic protein-specific CD4+ T cells that were transduced to express IL-12 p40 and luciferase. In vivo, luciferase was used to demonstrate trafficking to the central nervous system. In addition, IL-12 p40 inhibited inflammation. In another system, using positron emission tomography (PET), Koehne et al. (2003) demonstrated in vivo that Epstein-Barr virus (EBV)-specific T cells expressing herpes simplex virus-1 thymidine kinase (HSV-TK) selectively traffic to EBV+ tumors expressing the T cells' restricting HLA allele. Furthermore, these T cells retain their capacity to eliminate targeted tumors. Capitalizing on sequential imaging, Dubey et al. (2003) demonstrated antigen specific localization of T cells expressing HSV-TK to tumors induced by murine sarcoma virus/Moloney murine leukemia virus (M-MSV/M-MuLV). Tissue specific promoters may also be used to assess differentiation, for example, a stem cell differentiating or fusing with a liver cell and taking up the characteristics of the differentiated cell such as activation of the surfactant promoter in type II pneumocytes.

Preferably, a reporter sequence encodes a protein that is readily detectable either by its presence, its association with a detectable moiety or by its activity that results in the generation of a detectable signal. In certain aspects, a detectable moiety may include a radionuclide, a fluorophore, a luminophore, a microparticle, a microsphere, an enzyme, an enzyme substrate, a polypeptide, a polynucleotide, a nanoparticle, and/or a nanosphere, all of which may be coupled to an antibody or a ligand that recognizes and/or interacts with a reporter.

In various embodiments, a nucleic acid sequence of the invention comprises a reporter nucleic acid sequence or encodes a product that gives rise to a detectable polypeptide. A reporter protein is capable of directly or indirectly generating a detectable signal. Generally, although not necessarily, the reporter gene includes a nucleic acid sequence and/or encodes a detectable polypeptide that are not otherwise produced by the cells. Many reporter genes have been described, and some are commercially available for the study of gene regulation (e.g., Alam and Cook, 1990, the disclosure of which is incorporated herein by reference). Signals that may be detected include, but are not limited to color, fluorescence, luminescence, isotopic or radioisotopic signals, cell surface tags, cell viability, relief of a cell nutritional requirement, cell growth and drug resistance. Reporter sequences include, but are not limited to, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, G-protein coupled receptors (GPCRs), somatostatin receptors, CD2, CD4, CD8, the influenza hemagglutinin protein, symporters (such as NIS) and others well known in the art, to which high affinity antibodies or ligands directed thereto exist or can be produced by conventional means, and fusion proteins comprising a membrane bound protein appropriately fused to an antigen tag domain from, among others, hemagglutinin or Myc. Kundra et al., 2002, demonstrated noninvasive monitoring of somatostatin receptor type 2 chimeric gene transfer in vitro and in vivo using biodistribution studies and gamma camera imaging.

In some embodiments, a reporter sequence encodes a fluorescent protein. Examples of fluorescent proteins which may be used in accord with the invention include green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). It is to be understood that these examples of fluorescent proteins is not exclusive and may encompass later developed fluorescent proteins, such as any fluorescent protein within the infrared, visible or ultraviolet spectra.

In various embodiments, the desired level of expression of at least one of the reporter sequence is an increase, a decrease, or no change in the level of expression of the reporter sequence as compared to the basal transcription level of the diagnostic nucleic acid. In a particular embodiment, the desired level of expression of one of the reporter sequences is an increase in the level of expression of the reporter sequence as compared to the basal transcription level of the reporter sequence.

In various embodiments, the reporter sequence encodes unique detectable proteins which can be analyzed independently, simultaneously, or independently and simultaneously. In other embodiments, the host cell may be a eukaryotic cell or a prokaryotic cell. Exemplary eukaryotic cells include yeast and mammalian cells. Mammalian cells include human cells and various cells displaying a pathologic phenotype, such as cancer cells.

For example, some reporter proteins induce color changes in cells that can be readily observed under visible and/or ultraviolet light. The reporter protein can be any reporter protein known to those of ordinary skill in the art. Examples include gfp, rfp, bfp and luciferase.

Nucleic acids encoding reporter proteins include DNAs, cRNAs, mRNAs, and subsequences thereof encoding active fragments of the respective reporter amino acid sequence, as well as vectors comprising these sequences.

Exemplary methods of imaging of reporter proteins includes gamma camera imaging, CT, MRI, PET, SPECT, optical imaging, and ultrasound. In some embodiments, the diagnostic nucleic acid is suitable for imaging using more than one modality, such as CT and MRI, PET and SPECT, and so forth.

Additional information pertaining to examples of reporters in imaging are set forth in Kumar, 2005; Kundra et al., 2005; and Kundra et al., 2002, each of which is herein specifically incorporated by reference in its entirety.

### D. EXPRESSION CASSETTES

The therapeutic nucleic acid may itself be within an expression cassette. The term “expression cassette” is meant to include any type of genetic construct containing a nucleic acid coding for a gene product in which part or all of the nucleic acid encoding sequence is capable of being transcribed.

### 1. Promoters and Enhancers

In order for the expression cassette to effect expression of a transcript, the nucleic acid encoding gene will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a cell in any cell in a subject is contemplated as a promoter that can be applied with the methods of the present invention. In certain embodiments, for example, the promoter is a constitutive promoter, an inducible promoter, or a repressible promoter. The promoter can also be a tissue selective promoter. A tissue selective promoter is defined herein to refer to any promoter which is relatively more active in certain tissue types compared to other tissue types. Thus, for example, a liver-specific promoter would be a promoter which is more active in liver compared to other tissues in the body. One type of tissue-selective promoter is a tumor selective promoter. A tumor selective promoter is defined herein to refer to a promoter which is more active in tumor tissue compared to other tissue types. There may be some function in other tissue types, but the promoter is relatively more active in tumor tissue compared to other tissue types. Examples of tumor selective promoters include the hTERT promoter, the CEA promoter, the PSA promoter, the probasin promoter, the ARR2PB promoter, and the AFP promoter.

The promoter will be one which is active in the target cell. For instance, where the target cell is a keratinocyte, the promoter will be one which has activity in a keratinocyte. Similarly, where the cell is an epithelial cell, skin cell, mucosal cell or any other cell that can undergo transformation by a papillomavirus, the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. 2001, incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosine (melanoma), alpha-fetoprotein and albumin (liver tumors), CC10 (lung tumors) and prostate-specific antigen (prostate tumor) will permit tissue-specific expression of the therapeutic nucleic acids set forth herein. The following is a non-limited list of promoter/elements which may be employed in the context of the present invention: Immunoglobulin Heavy Chain, Immunoglobulin Light Chain, T-Cell Receptor, HLA DQ a and/or DQ β,β-Interferon, Interleukin-2, Interleukin-2 Receptor, MHC Class II 5, MHC Class II HLA-DRa, β-Actin, Muscle Creatine Kinase (MCK), Prealbumin (Transthyretin), Elastase I, Metallothionein (MTII), Collagenase, Albumin, α-Fetoprotein, t-Globin, β-Globin, c-fos, c-HA-ras, Insulin promoter, Neural Cell Adhesion Molecule (NCAM) promoter, α₁-Antitrypsin promoter, H2B (TH2B) Histone promoter, Mouse and/or Type I Collagen promoter, Glucose-Regulated Proteins (GRP94 and GRP78), Rat Growth Hormone, Human Serum Amyloid A (SAA), Troponin I (TN I), Platelet-Derived Growth Factor promoter, Polyomavirus promoters, Retrovirus promoters, Papilloma Virus promoters, Hepatitis B Virus promoters and Cytomegalovirus (CMV) promoters. This list is not intended to be exhaustive of all the possible promoter and enhancer elements, but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and continguous, often seeming to have very similar modular organization.

Additionally, any promoter/enhancer combination (as per the Eukaryotic Promoter Data Base EPDB) could also be used to drive expression of a gene. Use of a T3, T7, or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional expression vector.

Further selection of a promoter that is regulated in response to specific physiologic signals can permit inducible expression of a construct. For example, with the polynucleotide under the control of the human PAI-1 promoter, expression is inducible by tumor necrosis factor.

### 2. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

### 3. IRES

In certain instances, internal ribosome entry sites (IRES) elements may be incorporated into a nucleic acid to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each of which is herein incorporated by reference). One of ordinary skill in the art would be familiar with the application of IRES in gene therapy.

### 4. Multiple Cloning Sites

Nucleic acids can include a multiple cloning site (MCS), which is a region that contains multiple restriction enzyme sites, any of which can be used in conjunction with standard recombinant technology to digest the vector. See Carbonelli et al., (1999); Levenson et al., (1998); Cocea, (1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997).

### 5. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

### E. THERAPIES

### 1. Definitions

A “therapeutic nucleic acid” is defined herein to refer to a nucleic acid that is known or suspected to be of benefit in the treatment or prevention of a disease or health-related condition. Contemplated within the definition of “therapeutic nucleic acid” is a nucleic acid that encodes a protein or polypeptide that is known or suspected to be of benefit in the treatment of a disease or health-related condition. Therapeutic nucleic acids may also be nucleic acids which transcribe a nucleic acid that is known or suspected to be of benefit in the treatment of a disease or health-related condition (e.g., a nucleic acid transcribing a ribozyme). In the embodiments of this invention, a viral vector in a film or patch formulation may encode a therapeutic nucleic acid. In certain embodiments the therapeutic nucleic acid may be in a nucleic acid expression construct.

The term “therapeutic” or “therapy” as used throughout this application refers to anything that is known or suspected to promote or enhance the well-being of the subject with respect to a disease or health-related condition. Thus, a “therapeutic nucleic acid” is a nucleic acid that is known or suspected to promote or enhance the well-being of the subject with respect to a disease or health-related condition. A list of nonexhaustive examples of such therapeutic benefit includes extension of the subject's life by any period of time, or decrease or delay in the development of the disease. In the case of cancer, therapeutic benefit includes decrease in hyperproliferation, reduction in tumor growth, delay of metastases or reduction in number of metastases, reduction in cancer cell or tumor cell proliferation rate, decrease or delay in progression of neoplastic development from a premalignant condition, and a decrease in pain to the subject that can be attributed to the subject's condition.

A “disease” is defined as a pathological condition of a body part, an organ, or a system resulting from any cause, such as infection, genetic defect, or environmental stress.

A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

“Diagnostic” or “diagnosis” as used throughout this application refers to anything that is known or suspected to be of benefit in identifying the presence or absence of a disease or health-related condition in a subject. Also included in this definition is anything that is known or suspected to be of benefit in the identification of subjects at risk of developing a particular disease or health-related condition. Thus, a diagnostic nucleic acid is a nucleic acid that is known or suspected to be of benefit in identifying the presence or absence of a disease or health-related condition, or that is known or suspected to be of benefit in identifying a subject at risk of developing a particular disease or health-related condition. For example, the diagnostic nucleic acid may be a nucleic acid that encodes a reporter protein that is detectable. Such a protein, for example, may find application in imaging modalities.

An “effective amount” of the pharmaceutical composition, generally, is defined as that amount sufficient to detectably and repeatedly to achieve the stated desired result, for example, to ameliorate, reduce, minimize or limit the extent of the disease or its symptoms. More rigorous definitions may apply, including reduction in tumor growth rate, reduction in tumor size, inhibition of metastasis of primary tumor, inhibition of metastases (number or size, induction of apoptosis of cancer or tumor cells, sensitization to other cancer therapy such as radiotherapy or chemotherapy, prevention of recurrence, induction of remission, halting tumor growth, increased life span, or reduction in amount (courses and/or strength of doses) of other cancer therapy.

### 2. Diseases to be Diagnosed, Prevented or Treated

The present invention contemplates methods to detect, prevent, inhibit, or treat a disease in a subject by administration of a nucleic acid encoding an amino acid sequence capable of preventing or inhibiting disease in a subject. As set forth above, any nucleic acid sequence that can be applied or administered to a subject for the purpose of detecting, preventing, or inhibiting, or treating a disease is contemplated for inclusion in the pharmaceutical compositions set forth herein.

In certain embodiments, the disease may be a hyperproliferative disease that can affect a subject that would be amenable to detection, therapy, or prevention through administration of a nucleic acid sequence to the subject. For example, the disease may be a hyperproliferative disease. A hyperproliferative disease is a disease associated with the abnormal growth or multiplication of cells. The hyperproliferative disease may be a disease that manifests as lesions in a subject. Exemplary hyperproliferative lesions include the following: squamous cell carcinoma, basal cell carcinoma, adenoma, adenocarcinoma, linitis plastica, insulinoma, glucagonoma, gastrinoma, vipoma, cholangiocarcinoma, hepatocellular carcinoma, adenoid cystic carcinoma, carcinoid tumor, prolactinoma, oncocytoma, hurthle cell adenoma, renal cell carcinoma, endometrioid adenoma, cystadenoma, pseudomyxoma peritonei, Warthin's tumor, thymoma, thecoma, granulosa cell tumor, arrhenoblastoma, Sertoli-Leydig cell tumor, paraganglioma, pheochromocytoma, glomus tumor, melanoma, soft tissue sarcoma, desmoplastic small round cell tumor, fibroma, fibrosarcoma, myxoma, lipoma, liposarcoma, leiomyoma, leiomyosarcoma, myoma, myosarcoma, rhabdomyoma, rhabdomyosarcoma, pleomorphic adenoma, nephroblastoma, brenner tumor, synovial sarcoma, mesothelioma, dysgerminoma, germ cell tumors, embryonal carcinoma, yolk sac tumor, teratomas, dermoid cysts, choriocarcinoma, mesonephromas, hemangioma, angioma, hemangiosarcoma, angiosarcoma, hemangioendothelioma, hemangioendothelioma, Kaposi's sarcoma, hemangiopericytoma, lymphangioma, cystic lymphangioma, osteoma, osteosarcoma, osteochondroma, cartilaginous exostosis, chondroma, chondrosarcoma, giant cell tumors, Ewing's sarcoma, odontogenic tumors, cementoblastoma, ameloblastoma, craniopharyngioma gliomas mixed oligoastrocytomas, ependymoma, astrocytomas, glioblastomas, oligodendrogliomas, neuroepitheliomatous neoplasms, neuroblastoma, retinoblastoma, meningiomas, neurofibroma, neurofibromatosis, schwannoma, neurinoma, neuromas, granular cell tumors, alveolar soft part sarcomas, lymphomas, non-Hodgkin's lymphoma, lymphosarcoma, Hodgkin's disease, small lymphocytic lymphoma, lymphoplasmacytic lymphoma, mantle cell lymphoma, primary effusion lymphoma, mediastinal (thymic) large cell lymphoma, diffuse large B-cell lymphoma, intravascular large B-cell lymphoma, Burkitt lymphoma, splenic marginal zone lymphoma, follicular lymphoma, extranodal marginal zone B-cell lymphoma of mucosa-associated lymphoid tissue (MALT-lymphoma), nodal marginal zone B-cell lymphoma, mycosis fungoides, Sezary syndrome, peripheral T-cell lymphoma, angioimmunoblastic T-cell lymphoma, subcutaneous panniculitis-like T-cell lymphoma, anaplastic large cell lymphoma, hepatosplenic T-cell lymphoma, enteropathy type T-cell lymphoma, lymphomatoid papulosis, primary cutaneous anaplastic large cell lymphoma, extranodal NK/T cell lymphoma, blastic NK cell lymphoma, plasmacytoma, multiple myeloma, mastocytoma, mast cell sarcoma, mastocytosis, mast cell leukemia, langerhans cell histiocytosis, histiocytic sarcoma, langerhans cell sarcoma dendritic cell sarcoma, follicular dendritic cell sarcoma, Waldenstrom macroglobulinemia, lymphomatoid granulomatosis, acute leukemia, lymphocytic leukemia, acute lymphoblastic leukemia, acute lymphocytic leukemia, chronic lymphocytic leukemia, adult T-cell leukemia/lymphoma, plasma cell leukemia, T-cell large granular lymphocytic leukemia, B-cell prolymphocytic leukemia, T-cell prolymphocytic leukemia, pecursor B lymphoblastic leukemia, precursor T lymphoblastic leukemia, acute erythroid leukemia, lymphosarcoma cell leukemia, myeloid leukemia, myelogenous leukemia, acute myelogenous leukemia, chronic myelogenous leukemia, acute promyelocytic leukemia, acute promyelocytic leukemia, acute myelomonocytic leukemia, basophilic leukemia, eosinophilic leukemia, acute basophilic leukemia, acute myeloid leukemia, chronic myelogenous leukemia, monocytic leukemia, acute monoblastic and monocytic leukemia, acute megakaryoblastic leukemia, acute myeloid leukemia and myelodysplastic syndrome, chloroma or myeloid sarcoma, acute panmyelosis with myelofibrosis, hairy cell leukemia, juvenile myelomonocytic leukemia, aggressive NK cell leukemia, polycythemia vera, myeloproliferative disease, chronic idiopathic myelofibrosis, essential thrombocytemia, chronic neutrophilic leukemia, chronic eosinophilic leukemia/hypereosinophilic syndrome, post-transplant lymphoproliferative disorder, chronic myeloproliferative disease, myelodysplastic/myeloproliferative diseases, chronic myelomonocytic leukemia and myelodysplastic syndrome. In certain embodiments, the hyperproliferative lesion is a disease that can affect the mouth of a subject. Examples include leukoplakia, squamous cell hyperplastic lesions, premalignant epithelial lesions, intraepithelial neoplastic lesions, focal epithelial hyperplasia, and squamous carcinoma lesion.

In certain other embodiments, the hyperproliferative lesion is a disease that can affect the skin of a subject. Examples include squamous cell carcinoma, basal cell carcinoma, melanoma, papillomas (warts), and psoriasis. Treatment of carcinomas related to viruses is also contemplated, including but not limited to cancers of the head and neck. The lesion may include cells such as keratinocytes, epithelial cells, skin cells, and mucosal cells. The disease may also be a disease that affects the lung mucosa. In certain embodiments, the disease may be a precancerous lesion, such as leukoplakia of the oral cavity or actinic keratosis of the skin.

Other examples of diseases to be treated or prevented include infectious diseases and inflammatory diseases, such as autoimmune diseases. The methods and compositions of the present invention can be applied in to deliver an antigen that can be applied in immune therapy or immune prophylaxis of a disease.

### 3. Administration

The routes of administration will vary, naturally, with the location and nature of the lesion or site to be targeted, and include, e.g., intradermal, and oral administration.

Treatment regimens may vary as well, and often depend on tumor type, tumor location, immune condition, target site, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor or affected area being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor or targeted site.

The treatments may include various “unit doses.” Unit dose is defined as containing a predetermined-quantity of the therapeutic composition. The quantity to be administered is within the skill of those in the clinical arts. A unit dose need not be administered as a single dose but may comprise multiple doses over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher. Alternatively, the amount specified may be the amount administered as the average daily, average weekly, or average monthly dose.

The viral vectors are administered in a manner compatible with the dosage formulation, and in such amount as will be therapeutically effective. The quantity to be administered depends on the subject to be treated, including, e.g., the aggressiveness of the cancer, the size of any tumor(s), the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

### 4. Combination Treatments

In certain embodiments, the compositions and methods of the present invention involve a freeze-dried film strip or patch containing a viral vector and a secondary therapy, such as immunotherapy, radiotherapy or chemotherapy. These compositions would be provided in a combined amount effective to achieve the desired effect, for example, the killing of a cancer cell. This process may involve contacting the cells with the freeze-dried film strip or patch containing a viral vector and the secondary agent at the same or different times.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that the freeze-dried film or patch containing a viral vector may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example the freeze-dried film or patch formulation is “A” and the secondary therapy is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

c. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 1 is a non-limiting list of several known anti-cancer immunotherapeutic agents and their targets.

TABLE 1

| Generic Name | Target |
|---|---|
| cetuximab | EGFR |
| panitumumab | EGFR |
| trastuzumab | erbB2 receptor |
| bevacizumab | VEGF |
| alemtuzumab | CD52 |
| gemtuzumab ozogamicin | CD33 |
| rituximab | CD20 |
| tositumomab | CD20 |
| matuzumab | EGFR |
| ibritumomab tiuxetan | CD20 |
| tositumomab | CD20 |
| HuPAM4 | MUC1 |
| MORAb-009 | mesothelin |
| G250 | carbonic anhydrase IX |
| mAb 8H9 | 8H9 antigen |
| M195 | CD33 |
| ipilimumab | CTLA4 |
| HuLuc63 | CS1 |
| alemtuzumab | CD53 |
| epratuzumab | CD22 |
| BC8 | CD45 |
| HuJ591 | Prostate specific membrane antigen |
| hA20 | CD20 |
| lexatumumab | TRAIL receptor-2 |
| pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| volociximab | anti-a5β1 integrin |
| GC1008 | TGFβ |
| HCD122 | CD40 |
| siplizumab | CD2 |
| MORAb-003 | folate receptor alpha |
| CNTO 328 | IL-6 |
| MDX-060 | CD30 |
| ofatumumab | CD20 |
| SGN-33 | CD33 |

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient (Irie and Morton, 1986; Irie et al., 1989; Bajorin et al., 1988).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

d. Gene Therapy

In yet another embodiment, a combination treatment involves gene therapy in which a therapeutic polynucleotide is administered before, after, or at the same time as a therapeutic polypeptide, such as a tumor suppressor gene or nucleic acid encoding the therapeutic polypeptide. In other embodiments a gene therapy may be used in combination with a proteasome inhibitor. Delivery of a tumor suppressor polypeptide or encoding nucleic acid in conjunction with a vector encoding one of the following gene products, or the delivery of one of the following gene therapies combined with administration of a proteasome inhibitor may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of a Film or Patch for Topical Gene Therapy Application

### Introduction

A film or patch is preferred for topical delivery of gene therapy products to the oral cavity. A number of biopolymers were evaluated for compatibility with adenoviral vectors. Biopolymer solutions containing adenovirus must be dried in order to form a film.

### Materials and Methods

a) Biopolymers
Hydroxypropylmethyl cellulose (HPMC): Sigma H7509-100G, Batch #105K0158
Hydroxypropyl cellulose (HPC): Acros Cat# 184882500, Lot#A0203768001
Sodium alginate: Sigma Cat# A2033-100G, Batch#035K0205

b) Adenovirus
A replication defective serotype 5 adenovirus encoding wild type p53 was used.

c) Water
WFIr: B. Braun, Cat# R5007, Lot#J6A227

Hydroxypropylmethyl cellulose, hydroxypropyl cellulose or sodium alginate were dissolved in WFIr to make solutions of 1%, 5% and 1% (w/v) respectively. Approximately 1 ml of each of the solutions was mixed with 1 ml of the adenoviral vector in solution to form a mixture containing adenovirus. The solutions were maintained at room temperature for 5 minutes. After this time period, a portion of each solution was analyzed by ion exchange HPLC for a determination of virus particle concentration. The results are shown in table 1. The remaining portion of the adenovirus biopolymer mixtures were placed at 37° C. for a preliminary stability study. Daily samples were taken for ion exchange HPLC analysis. The results are shown in table 2.

TABLE 1

| | | Expected titer in the |
|---|---|---|
| Sample | HPLC titer (vp/mL) | mixture |
| Ad5/l% HPMC | 6E11 | 5E11 |
| Ad5/5% HPC | 5E11 | 5E11 |
| Ad5/l% NaAlginate | 7E11 | 5E11 |

TABLE 2

| Day of storage | HPLC titer (vp/mL) | | |
|---|---|---|---|
| at 37° C. | Ad5/1% HPMC | Ad5/5% HPC | Ad5/1% NaAlginate |
| 0 | 6E11 | 5E11 | 7E11 |
| 1 | 4.6E11 | 1.7E11 | 7.5E11 |
| 2 | None detected | None detected | None detected |

### Results and Discussion

Based on the HPLC analysis of virus particle determination, it was found that the adenovirus maintained integrity after initial mixing with all three biopolymer solutions. As a result, all the biopolymers appear to be initially compatible with adenovirus. However, as shown in table 2, the adenovirus titer rapidly decreased over a period of days in storage at 37° C., culminating with no virus detected on the second day. However, the data suggests that adenovirus is more stable in the 1% sodium alginate solution. As a result, Sodium Alginate was chosen for further testing.

### Example 2

### Air Drying of an Adenoviral Vector Containing Film

### Materials and Methods

The adenoviral vector/1% sodium alginate solution (0.3 ml) was placed into a well of a 12 well plate to cover the bottom of the well. The material was allowed to dry for one day. The next day, a thin film was formed on the bottom of the well. The film was re-constituted with 0.3 mL of WFIr. A sample of the re-constituted solution was analyzed by the HPLC.

### Results and Discussion

No virus was detected by the HPLC, suggesting most of the virus lost integrity during the air drying process. This is not surprising based on our previous experience that adenovirus is very sensitive to air drying.

### Example 3

### Freeze Drying of an Adenoviral Vector Containing Film

### Introduction

Based on our previous lack of success in maintaining the integrity of adenoviral vectors in an alginate film during a regular drying process, freeze-drying was evaluated as an alternative choice to produce a biopolymer film containing functional adenovirus.

### Materials and Methods

A 2% sodium alginate solution was prepared by dissolving 1 g of sodium alginate in 50 ml of WFIr. 2.2 ml of this solution was mixed with 2.2 ml of the p53 wild type containing adenoviral vector (Ad5 wt virus 207-028) to produce a mixture containing 1% sodium alginate and approximately 6×10¹¹ vp/ml of adenovirus.

The solution was transferred in either 1 or 2 ml increments into separate wells of a 6 well plate and placed on a Dura-Stop mp freeze-dryer shelf (FTS Systems, Stone Ridge, N.Y.). The material was freeze-dried by a manual program, briefly:
1. Cool the shelf to −30° C. to freeze the mixture;
2. Upon freezing, initiate the drying process by vacuum pump; and
3. Allow the shelf temperature to gradually increase to 10° C. before removing the 6 well plate from the freeze-drier.

### Results and Discussion

A thin film was formed for both the 1 ml and 2 ml load conditions. The 2 ml load film was re-constituted using 2 ml WFIr. A sample was analyzed by HPLC. Unfortunately, no virus was detected in the re-constituted solution suggesting most of the virus lost integrity during the freeze drying process.

### Example 4

### Freeze Drying of an Adenoviral Vector Containing Film With Lyoprotectant

### Introduction

The loss of virus integrity in Experiment 1 may be related to the fact that no lyo-protectant was included in the freeze drying process and freeze drying cycle was not appropriate. In this experiment sucrose was included as a lyo-protectant and the freeze drying cycle was modified.

### Materials and Methods

A 2% sodium alginate solution was prepared by dissolving 1 g of sodium alginate in 50 ml of WFIr. 5 g of sucrose as a lyoprotectant was added to the solution to produce a 2% sodium alginate solution containing 10% sucrose. 2.2 ml of the solution was mixed with 2.2 ml of the p53 wild type containing adenoviral vector (Ad5 wt virus 207-028) to produce a mixture containing 1% sodium alginate+5% sucrose and approximately 6×10¹¹ vp/ml of adenovirus.

The solution was transferred in either 1 or 2 ml increments into separate wells of a 6 well plate and placed on a Dura-Stop mp freeze-dryer shelf (FTS Systems, Stone Ridge, N.Y.). The material was freeze-dried by a manual program, briefly:
1. The shelf was pre-cooled to −40° C.;
2. the 6 well plate was placed on the pre-cooled shelf to freeze the material;
3. After the mixture was frozen (at −37° C.), the drying process was initiated by vacuum pump (100 mT);
4. The frozen solution in the 6 well plates was allowed to dry at −37° C. for 6 hours;
5. After 6 hours of drying the shelf temperature was increased to 0° C.; and
6. The film was dried briefly at 0° C. before removal from the freeze drier.

### Results and Discussion

A thin film was formed for both the 1.0 ml and the 1.5 ml load conditions. No film shrinkage was observed during the drying process. The 1.0 ml load film was re-constituted using 1 ml of WFIr and analyzed by HPLC. The results are shown in table 3. In contrast to the results without the use of a lyoprotectant, no loss of adenovirus integrity occurred during the freeze drying process. The result suggests that adenovirus can be formed into a dry film in association with a biopolymer such as sodium alginate.

TABLE 3

| Sample | HPLC titer (vp/mL) |
|---|---|
| Pre-freeze dry solution | 7.5E11 |
| Re-constituted dry film solution | 8.2E11 |

### Example 5

### Freeze Drying of an Ad-GFP Film

### Materials and Methods

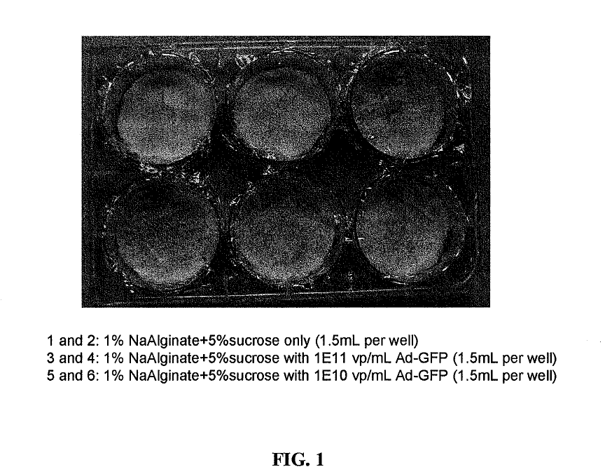

A film was produced by freeze drying and adenoviral vector containing the green fluorescent protein gene (Ad-GFP) in a 1% (working) sodium alginate and a 5% (working) sucrose solution. The Ad-GFP was formulated in 20 mM Tris-HCL and 10% glycerol, pH 8.20. Equal volume of Ad-GFP stock solution was mixed with a 2% sodium alginate+10% sucrose solution. 1.5 mL of the virus mixture was added to each well of a 6-well plate with either 1×10¹⁰ or 1×10¹¹ vp/ml of virus. A solution of 1% sodium alginate and 5% sucrose served as a control. The wells of the 6-well plate were first lined with a food wrapping film as a backing film. FIG. 1. The material was freeze dried using a FTS systems freeze dryer. The drying cycle steps are as follows:
1. Freeze to −40° C.
2. Initiate vacuum at a set point of 200 mT after the material temperature dropped to −40° C.
3. Allow to dry at −40° C. overnight
4. Decrease the vacuum set point to 100 mT
5. 1 hour later, increase the temperature to −30° C.
6. 1 hour later, increase the temperature to −20° C.
7. 1 hour later, increase the temperature to −10° C.
8. 1 hour later, increase the temperature to −0° C.
9. 1 hour later, increase the temperature to 10° C.
10. 1 hour later, increase the temperature to 15° C.
11. 1 hour later, stop the drying cycle.
   Following the freeze drying procedure, the films were removed from the wells and were cut into small pieces for both HPLC analysis and 293 cell transduction analysis. For 293 cell transduction analysis, a piece of the cut film was placed on top of a 293 cell monolayer with the film facing down and the food wrapping film facing up. Subsequently culture media was added and the cells were incubated with the film for a period of one hour at which time the film was removed and the cells were incubated overnight at 37° C. Following overnight incubation cells were observed for GFP expression via fluorescence microscope.

### Results and Discussion

It appears that the presence of 1% glycerol in the 1×10¹¹ vp/ml condition (1:10 diluted from the Ad-GFP virus stock) helped to form a flexible film after freeze drying. FIG. 1. The other films were somewhat brittle.

HPLC analysis of the film showed approximately 90% virus recovery for the 1×10¹¹ vp/ml sample (Data Not Shown).

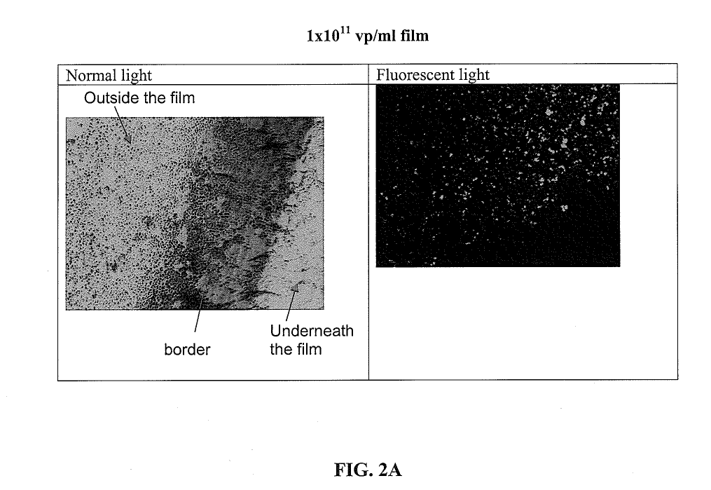

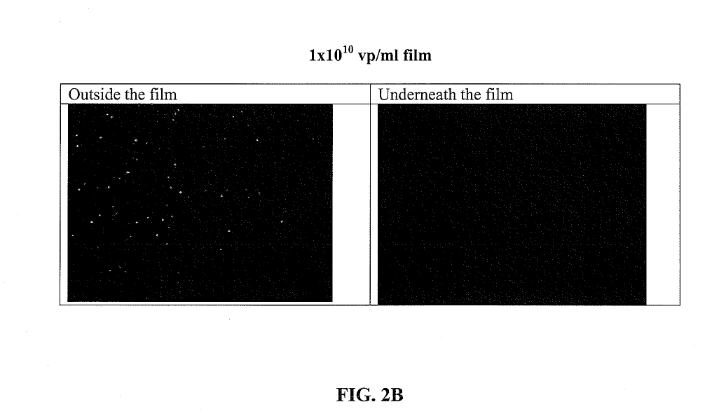

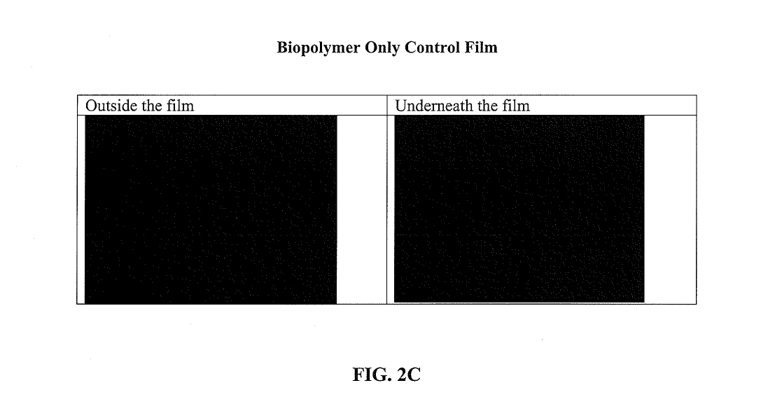

Ad-GFP transduction analysis demonstrated GFP expression for both the 1×10¹¹ and 1×10¹⁰ vp/ml samples. Almost all the cells in the 1×10¹¹ vp/ml sample were expressing GFP. FIG. 2A. One interesting observation was that the cells underneath the film were not healthy and were not expressing GFP. Because the tight association of the gel formed from the film, it is suspected that the cells immediately underneath the film were likely to be no longer viable due to lack of oxygenation. As in the case of the 1×10¹¹ vp/ml sample, the cells from the 1×10¹⁰ vp/ml sample also expressed GFP, albeit at a lower level, thus indicating a dose response effect. FIG. 2B. No GFP expression was observed in the biopolymer only control film condition. FIG. 2C.

### Example 6

### Freeze Dried Film Stability Assay

### Materials and Methods

A freeze drying of Ad-GFP virus in 1% Sodium Alginate+5% sucrose was carried as described before. A total of 6 wells of films were prepared in 6-well plates. The films were subjected to freeze-drying as describe previously for a period of 24 hours. Following the freeze-drying procedure, the films were removed from each well. Each film was cut in half. One half was used for time 0 testing by HPLC and transduction on 293 cells grown in 6 well plates. The remaining films were placed individually in foil pouches and sealed. (Ampac Packaging, Cincinnati, Ohio). All films were frozen and stored at −20° C. HPLC analysis is shown in Table 4 below.

TABLE 4
HPLC Analysis of Films After Storage

| Storage time at | |
|---|---|
| -20° C. (months) | HPLC titer |
| 0 | 1.7 × 10¹¹ vp/ml |
| 1 | 2.3 × 10¹¹ vp/ml |
| 2 | 1.7 × 10¹¹ vp/ml |

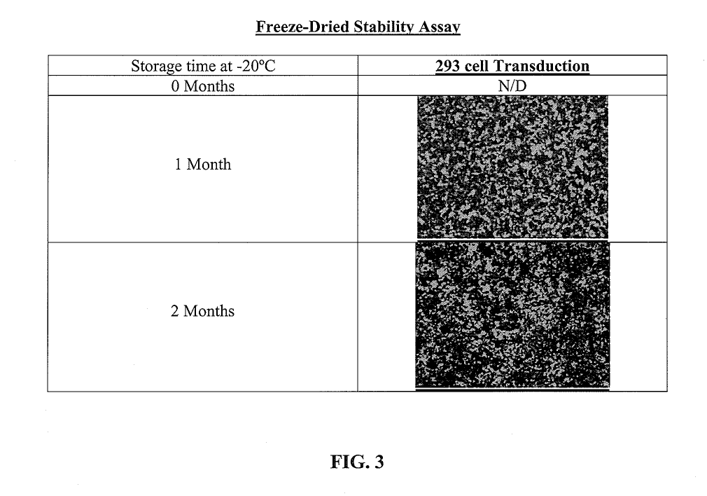

In addition the freeze dried films were tested for transduction efficiency using 293 target cells. FIG. 3. The results are consistent with the HPLC analysis.

### Example 7

### Transduction Efficiency Based on Film Contact Time

### Materials and Methods

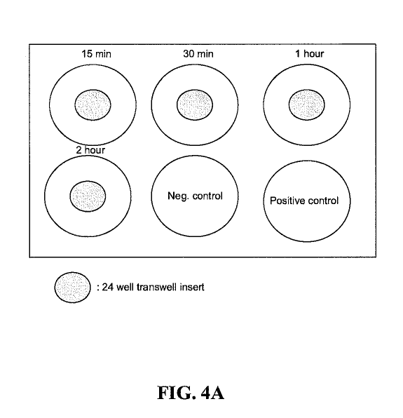

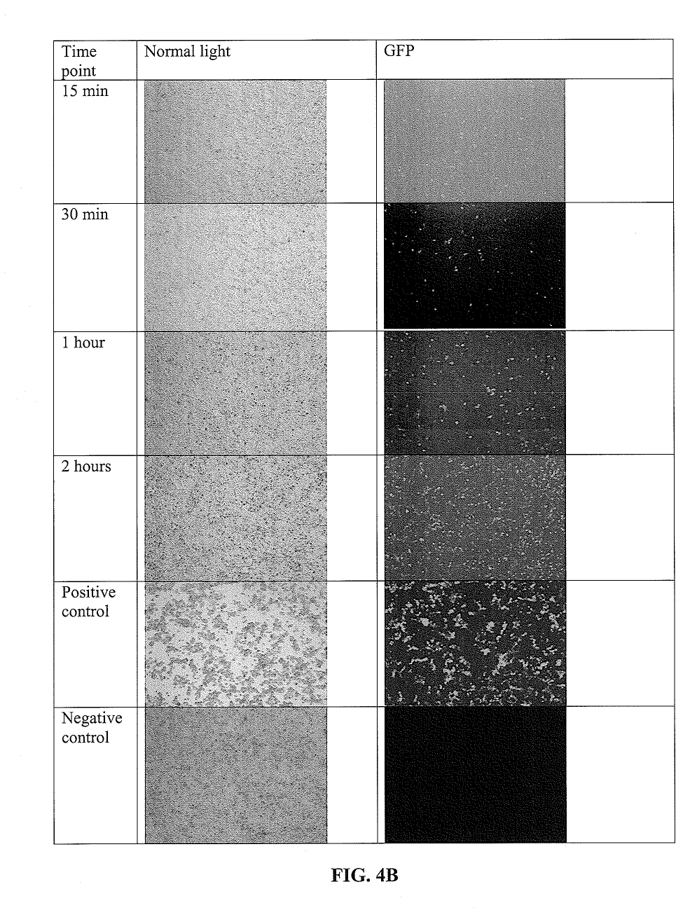

Freeze dried Ad-GFP film as described in Example 6 was used in this study. Briefly, the film, which was stored at −20° C. for 14 days, was used to transduce 293 target cells. Target 293 cells were seeded into 6 well plates in order to form a confluent monolayer one day prior to transduction by freeze-dried Ad-GFP film. The following day, media was aspirated from the 293 cells and a 24-well trans-well insert (3 μm membrane) was placed on top of the cell monolayer. A small piece of the Ad-GFP freeze dried film was placed inside the 24-well insert. 1 ml of media was added to the well, of each 6 well plate and 0.25 ml of media was added to the inside of the 24-well insert to ensure that the film was wetted. Following contact of the film with the 293 target cells, the 6 well plate was incubated at 37° C. At different time points post incubation, a 24-well insert with the film inside was removed from the cell monolayer. Following removal of the 24-well insert at specified time points, an additional 1 ml of media was added to the well of the 6 well plate from which the 24 well insert had been removed. Following removal of all 24-well inserts, the 6 well plate was allowed to incubate overnight at 37° C. overnight for GFP observation on the next day. For positive control, a film of a similar size was placed directly on top of the cell monolayer and was not removed until GFP observation. The well layout is shown in FIG. 4A.

### Results and Discussion

Target 293 cells were kept in contact with Ad-GFP-freeze dried film for intervals of 15 minutes, 30 minutes, 1 hour or 2 hours prior to removal of the film from the target cells and addition of 1 ml of media. Following overnight incubation at 37° C., cells were observed for GFP expression normal and ultraviolet light. Length of exposure of the 293 target cells to the Ad-GFP film strongly correlated with subsequent GFP expression the following day as shown in FIG. 4B.

### Example 8

### Transduction Efficiency of Freeze-Dried Ad-GFP Film in Oral Epithelial Model

### Introduction

Following demonstration of transduction efficiency of freeze dried Ad-GFP film in 293 cells, the inventors sought to apply this technique to an oral epithelial cell culture model. For this experiment fresh freeze-dried Ad-GFP film as described in Example 6 was used.

### Materials and Methods

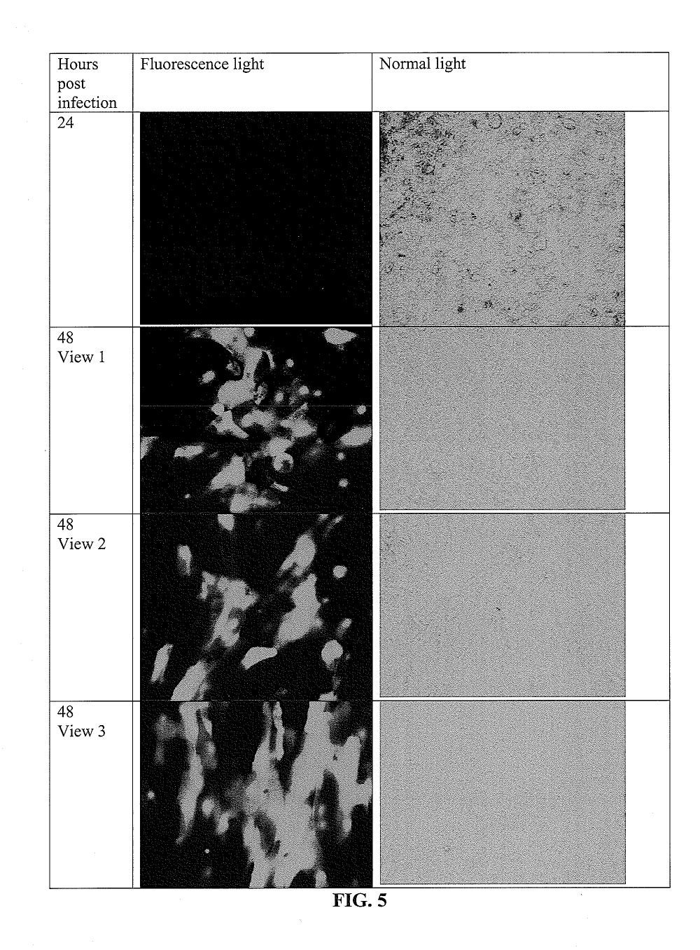

EpiOral™ cells (MatTek Corporation, Ashland, Mass.) in the form of a tissue insert were transferred to 6 well plates. Assuming a cell number of 1×10⁶ cells in the tissue insert, the cells were transduced with fresh freeze-dried Ad-GFP film as described in Experiment 6. The film was placed on the apical surface of the EpiOral™ culture and remained on the culture for the duration of the experiment. Cells were observed for GFP expression at 24 hours post freeze-dried film exposure and 48 hours post freeze-dried film exposure. As shown in FIG. 5, no GFP expression was observed after an exposure of 24 hours whereas GFP expression from the EpiOral™ culture was observed after 48 hours.

### Example 9

### Ad-GFP Labeling of H1299 Tumor Cells in Oral Epithelial Model

### Introduction

Following demonstration of transduction efficiency of freeze-dried Ad-GFP film at 48 hours on the apical surface of the EpiOral™ oral epithelial model, the inventors sought to test detection of a neoplastic tissue in an oral model. H11299 non small cell lung cancer cells were selected as a neoplastic target for use in the oral epithelial model.

### Materials and Methods

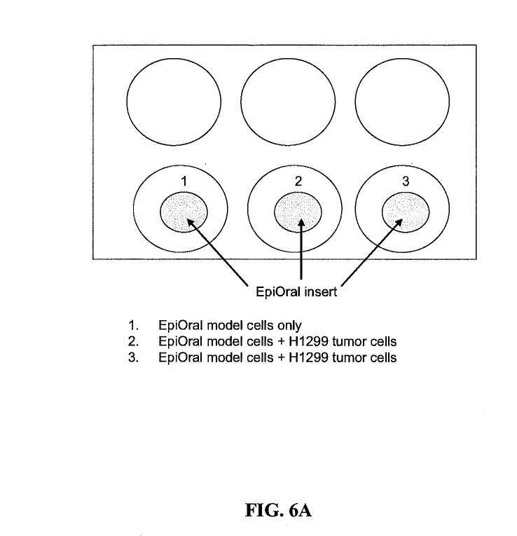

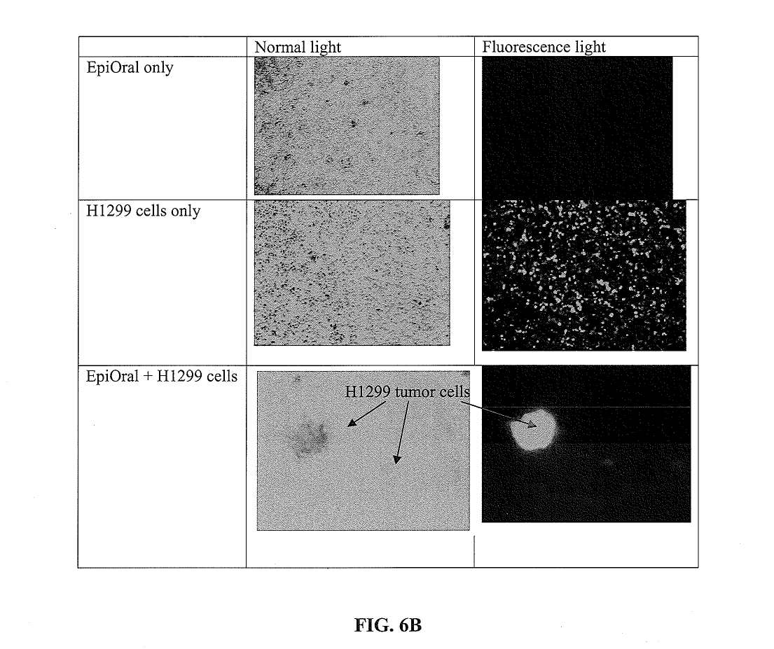

Three wells from a 24 well plate of EpiOral™ cells (MatTek Corporation, Ashland, Mass.) were removed from the 24 well plate and added to 3 wells of a 6 well plate. 2 ml of media provided by MatTek was added to these three wells containing the EpiOral™ cells. Subsequently, 50 μl of trypsinized H1299 cells (7.1×10⁴ cells) was placed on top of the EpiOral™ cell monolayer in two of the three wells. FIG. 6A. A small piece of AD-GFP film as described in Example 6 was placed on top of the cell monolayer of each of the three wells. To help hydrate the film, 50 μl of media was added to each well. The 6 well plate was incubated for a period of 24 hours at 37° C. As a control, H1299 cells plated in another 6-well plate were transduced using the same Ad-GFP film as described above. At the end of the incubation period the cells were observed under fluorescence microscope for GFP Expression. FIG. 6B.

### Results and Discussion

As shown in FIG. 8B, at 24 hours post application of the Ad-GFP film, only the H1299 cancer cells can be seen under the fluorescence microscope. Almost no EpiOral™ cells were expressing GFP at this time point. Based on our previous studies, it took at least 48 hours post application of the Ad-GFP film for some of the cells in the EpiOral™ monolayer to express GFP. These results indicate the possibility of exploiting the different adenovirus infection and expression kinetics between normal oral cells and tumor cells to achieve selective labeling (diagnosis) of cancerous cells in the oral cavity.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
PCT Application WO0208436
PCT Application WO0231168
PCT Application WO0285287
PCT Application WO0333029
U.S. Pat. No. 4,683,202
U.S. Pat. No. 4,797,368
U.S. Pat. No. 4,946,778
U.S. Pat. No. 5,139,941
U.S. Pat. No. 5,571,314
U.S. Pat. No. 5,672,344
U.S. Pat. No. 5,770,219
U.S. Pat. No. 5,783,208
U.S. Pat. No. 5,888,493
U.S. Pat. No. 5,888,773
U.S. Pat. No. 5,914,334
U.S. Pat. No. 5,925,565
U.S. Pat. No. 5,928,906
U.S. Pat. No. 5,935,819
U.S. Pat. No. 6,258,830
U.S. Pat. No. 6,280,752
U.S. Pat. No. 6,280,766
U.S. Pat. No. 6,348,450
U.S. Pat. No. 6,555,131
U.S. Pat. No. 6,627,190
U.S. Pat. No. 6,828,308
U.S. patent application Ser. No. 09/351,778
U.S. patent application Ser. No. 10/383,266
U.S. patent application Ser. No. 10/741,861
U.S. patent application Ser. No. 10/810,063
U.S. patent application Ser. No. 11/336,664
Alam and Cook, Anal. Biochem., 188:245-254, 1990.
Caley et al., J Virology, 71(4):3031-3038, 1997.
Carbonelli et al., FEMS Microbiol. Lett., 177(1):75-82, 1999.
Chandler et al., Proc. Natl. Acad. Sci. USA, 94(8):3596-601, 1997.
Chang et al., Hepatology, 14:134A, 1991.
Clark et al., Hum. Gene Ther., 6(10):1329-1341, 1995.
Clayman et al., Cancer Res., 55(14):1-6, 1995.
Cocea, Biotechniques, 23(5):814-816, 1997.
Coffin, In: Virology, Fields et al. (Eds.), Raven Press, NY, 1437-1500, 1990.
Costa et al., Mol. Cell. Biol., 8:81, 1988.
Couch et al., Am. Rev. Resp. Dis., 88:394-403, 1963.
Dalgleish, Expert Rev. Vaccines, 3(6):665-668, 2004.
Davis et al, Curr. Biol., 6:146-148, 1996.
DeLuca et al., J. Virol., 56(2):558-570, 1985.
Devulapalle and Mooser, J. Dent. Res., 80(2):466-469, 2001.
Dubey et al., Proc. Natl. Acad. Sci. USA, 100(3):1232-1237, 2003.
Eicher et al., Clin. Cancer Res., 2(10):1659-1664, 1996.
Elroy-Stein et al., Proc. Natl. Acad. Sci. USA, 86(16):6126-6130, 1989.
Finn et al., Cancer Chemother. Biol. Response Modif, 21:223-233, 2003.
Flotte and Carter, Gene Ther., 2(6):357-62, 1995.
Flotte et al., Proc. Natl. Acad. Sci. USA, 90(22):10613-10617, 1993.
Flotte et al., Am. J. Respir. Cell Mol. Biol., 7(3):349-356, 1992.
Ghosh-Choudhury et al., EMBO J, 6:1733-1739, 1987.
Glorioso et al., Mol. Biotechnol., 4(1):87-99, 1995.
Gomez-Foix et al., J. Biol. Chem., 267:25129-25134, 1992.
Graham and Prevec, In: Methods in Molecular Biology: Gene Transfer and Expression Protocol, Murray (Ed.), Humana Press, NJ, 7:109-128, 1991.
Graham and Prevec, 1992)
Graham et al., J. Gen. Virl., 36(1):59-74, 1977.
Grunhaus and Horwitz, Seminar in Virology, 3:237-252, 1992.
Guo et al., J. Dent. Res., 83(3):266-270, 2004.
Hellstrom and Hellstrom, Expert Rev. Vaccines, 2(4):517-532, 2003.
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Holland and Holland, J Biol Chem, 255(6):2596-605, 1980.
Honess and Roizman, J. Virol., 14(1):8-19, 1974.
Honess and Roizman, J. Virol., 16(5):1308-130826. 1975.
Horwich, et al., J. Virol., 64:642-650, 1990.
Jones and Shenk, Cell, 13:181-188, 1978.
Kaplitt et al., Nat Genet., 8(2):148-154, 1994.
Karlsson et al., EMBO J., 5:2377-2385, 1986.
Koehne et al., Nat. Biotechnol., 21(4):405-413, 2003.
Kotin et al., Proc. Natl. Acad. Sci. USA, 87(6):2211-2215, 1990.
Kumar, 2005;
Kundra et al., J. Nucl. Med., 43(3):406-412, 2002.
Kundra et al., Q J Nucl. Med. Mol. Imaging, 49(4):325-338.2005.
LaFace et al., Virology, 162(2):483-486, 1988.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levenson et al., Hum. Gene Ther., 9(8):1233-1236, 1998.
Levrero et al., Gene, 101:195-202, 1991.
Linchey and Fridovich, Free Radic. Biol. Med., 23(4):668-671, 1997.
Macejak and Samow, Nature, 353:90-94, 1991.
Mann et al., Cell, 33:153-159, 1983.
Markowitz et al., J. Virol., 62:1120-1124, 1988.
McCarty et al., J. Virol., 65(6):2936-2945, 1991.
McLaughlin et al., J. Virol., 62(6):1963-1973, 1988.
Muzyczka, Curr. Topics Microbiol. Immunol., 158:97-129, 1992.
Nicolas and Rubenstein, In: Vectors. A survey of molecular cloning vectors and their uses, Rodriguez and Denhardt (Eds.), Stoneham: Butterworth, 494-513, 1988.
Ohi et al., Gene, 89(2):279-282, 1990.
Paskind et al., Virology, 67:242-248, 1975.
Pelletier and Sonenberg, Nature, 334(6180):320-325, 1988.
Racher et al., Biotechnology Techniques, 9:169-174, 1995.
Ragot et al., Nature, 361:647-650, 1993.
Renan, Radiother. Oncol., 19:197-218, 1990.
Rich et al., Hum. Gene Ther., 4:461-476, 1993.
Rosenfeld et al., Science, 252:431-434, 1991.
Rosenfeld, et al., Cell, 68:143-155, 1992.
Sambrook et al., In: Molecular cloning, Cold Spring Harbor Laboratory Press, Cold Spring Harbor, N.Y., 2001.
Samulski et al., J. Virol., 63:3822-3828, 1989.
Samulski et al., EMBO J, 10:3941-3950, 1991.
Shelling and Smith, Gene Therapy, 1:165-169, 1994.
Siegfried, Exp. Clin. Endocrinol., 101(1):7-11, 1993.
Smith and Moss, Gene, 25(1):21-8, 1983.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Straus et al., J. Cell Biol., 99(5):1838-1847, 1984.
Tang et al., Blood, 104(9):2704-2713, 2004.
Temin, In: Gene Transfer, Kucherlapati (Ed.), NY, Plenum Press, 149-188, 1986.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Walsh et al., J. Clin. Invest, 94:1440-1448, 1994.
Wei et al., Gene Therapy, 1:261-268, 1994.
Wheeler et al., Gastrointerology, 120:1241-1250, 2001
Wheeler et al., Hum. Gene Ther., 12:2167-2177, 2001
Yang et al., J. Virol., 68:4847-4856, 1994.
Yoder et al., Blood, 82 (Supp.): 1:347A, 1994.
Zhou et al., Exp. Hematol, 21:928-933, 1993.
Zhou et al., J. Exp. Med., 179:1867-1875, 1994.

1. A method of producing a film, strip, or patch for delivery of a viral vector to a subject, comprising:
(a) casting a composition comprising a viral vector, a biopolymer, and an aqueous solvent into a mold,
(b) drying the composition in the mold to remove some or all of the aqueous solvent, and
(c) removing the dried composition of (b) from the mold,
wherein a film, strip, or patch is formed.

2. The method of claim 1, wherein the biopolymer is selected from the group consisting of hydroxypropylmethyl cellulose, hydroxypropyl cellulose, sodium alginate, and polyacrylate.

3. The method of claim 2, wherein the biopolymer is sodium alginate.

4. The method of claim 1, wherein the aqueous solvent is water.

5. The method of claim 4, wherein the weight to volume ratio of biopolymer to water in the composition of (a) is about 0.1% to about 15%.

6-7. (canceled)

8. The method of claim 1, wherein the composition of (a) further comprises a lyoprotectant.

9. The method of claim 8, wherein the lyoprotectant is selected from the group comprising sucrose, fructose, glucose, galactose, mannose, sorbitol, trehalose, lactose, maltose, and mannitol.

10. (canceled)

11. The method of claim 8, wherein the aqueous solvent is water, and the weight to volume ratio of lyoprotectant to water in the composition of (a) is about 1% to about 20%.

12-17. (canceled)

18. The method of claim 1, wherein the composition of (a) further comprises a buffer.

19. The method of claim 18, wherein the buffer is Tris-HCL, TES, HEPES, mono-Tris, brucine tetrahydrate, EPPS, tricine or histidine.

20-22. (canceled)

23. The method of claim 1, wherein the viral vector is an adenoviral vector, an adeno-associated viral vector, a retroviral vector, a herpesviral vector or a pox viral vector.

24. The method of claim 23, wherein the viral vector is an adenoviral vector.

25. The method of claim 1, wherein drying the composition in the mold comprises freeze-drying the composition in the mold.

26. The method of claim 1, further comprising cutting the dried composition into films, strips, or patches following removal of the dried composition from the mold.

27. The method of claim 25, further comprising storing the film, strip, or patch at about 4° C. to about −80° C. after freeze-drying.

28-43. (canceled)

46. The method of claim 1, wherein the viral vector comprises a therapeutic nucleic acid.

47. The method of claim 46, wherein the therapeutic nucleic acid encodes a tumor suppressor or a tumor antigen.

48. The method of claim 47, wherein the tumor suppressor is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a SEM A3 polypeptide.

49. The method of claim 48, wherein the tumor suppressor is p53, mda7, or FUS1.

50. The method of claim 47, wherein the tumor antigen is MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, ING1, mamaglobin, cyclin B1, S100, BRCA1, BRCA2, a tumor immunoglobulin idiotype, a tumor T-cell receptor clonotype, MUC-1, or epidermal growth factor receptor.

51. The method of claim 46, wherein the therapeutic nucleic acid is comprised in an expression cassette comprising a promoter operatively coupled to the nucleic acid, wherein the promoter is active in cells of a subject.

52. The method of claim 30, wherein the viral vector comprises a diagnostic nucleic acid.

53. The method of claim 52, wherein the diagnostic nucleic acid encodes a fluorescent protein selected from the group consisting of blue fluorescent protein, green fluorescent protein, yellow fluorescent protein, red fluorescent protein, or cyan fluorescent protein.

54. A film, strip, or patch for delivery of a viral vector to a subject, comprising:
i) a biopolymer
ii) a lyoprotectant; and
iii) a viral vector.

55. The film, strip, or patch of claim 54, wherein the biopolymer is selected from the group consisting of hydroxypropylmethyl cellulose, hydroxypropyl cellulose, and sodium alginate.

56. The film, strip, or patch of claim 54, wherein the lyoprotectant is selected from the group consisting of sucrose, fructose, glucose, galactose, mannose, sorbitol, trehalose, lactose, maltose, and mannitol.

57-59. (canceled)

60. The film, strip, or patch of claim 54, wherein the viral vector is an adenoviral vector, an adeno-associated viral vector, a retroviral vector, a herpesviral vector or a pox viral vector.

61. The film, strip, or patch of claim 60, wherein the viral vector is an adenoviral vector.

62. The film, strip, or patch of claim 54, wherein the viral vector comprises a therapeutic nucleic acid.

63. The film, strip, or patch of claim 62, wherein the therapeutic nucleic acid encodes a tumor suppressor or a tumor antigen.

64. The film, strip, or patch of claim 63, wherein the tumor suppressor is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a SEM A3 polypeptide.

65. The film, strip, or patch of claim 64, wherein the tumor suppressor is p53, mda7, or FUS1.

66. The film, strip, or patch of claim 63, wherein the tumor antigen is MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, p185erbB2, p180erbB-3, c-met, mn-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, INGI, mamaglobin, cyclin B1, S100, BRCA1, BRCA2, a tumor immunoglobulin idiotype, a tumor T-cell receptor clonotype, MUC-1, or epidermal growth factor receptor.

67. The film, strip, or patch of claim 62, wherein the therapeutic nucleic acid is comprised in an expression cassette comprising a promoter operatively coupled to the nucleic acid, wherein the promoter is active in cells of a subject.

68. The film, strip, or patch of claim 54, wherein the viral vector comprises a diagnostic nucleic acid.

69. The film, strip, or patch of claim 68, wherein the diagnostic nucleic acid encodes a fluorescent protein selected from the group consisting of blue fluorescent protein, green fluorescent protein, yellow fluorescent protein, red fluorescent protein, and cyan fluorescent protein.

70-86. (canceled)

87. A method of detecting, treating or preventing disease in a human subject, comprising applying to a body surface of a subject a film, strip, or patch comprising
i) a biopolymer
ii) a lyoprotectant;
iii) a viral vector, wherein the viral vector comprises a therapeutic or diagnostic nucleic acid.

88-90. (canceled)

91. The method of claim 90, wherein the disease is cancer.

92. The method of claim 91, wherein the cancer is breast cancer, lung cancer, prostate cancer, ovarian cancer, brain cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

93. The method of claim 87, wherein administering comprises applying the strip or patch to a mucosal surface of the subject.

94. The method of claim 93, wherein the mucosal surface is a surface of the oral cavity of the subject.

95. The method of claim 87, wherein the viral vector comprises a therapeutic nucleic acid that encodes a tumor suppressor.

96. The method of 95, wherein the tumor suppressor is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a SEM A3 polypeptide.

97. The method of claim 96, wherein the tumor suppressor is p53, MDA-7, or FUS1.

98-99. (canceled)
